(19) United States
(12) Patent Application Publication
(10) Pub. No.: US 2025/0205233 A1
(43) Pub. Date: Jun. 26, 2025

(54) EPIDERMAL GROWTH FACTOR RECEPTOR (EGFR) TYROSINE KINASE INHIBITORS IN COMBINATION WITH AN AKT INHIBITOR FOR THE TREATMENT OF CANCER

(71) Applicant: AstraZeneca AB, Sodertalje (SE)

(72) Inventors: Nicolas FLOCH, Cambridge (GB); Matthew Joseph MARTIN, Cambridge (GB); Paul David SMITH, Cambridge (GB); Ursula GRAZINI, Cambridge (GB)

(21) Appl. No.: 18/851,158

(22) PCT Filed: Mar. 30, 2023
(86) PCT No.: PCT/EP2023/058261

§ 371 Date: Sep. 26, 2024

Related U.S. Application Data
(60) Provisional application No. 63/325,861, filed on Mar. 31, 2022.

(51) Int. Cl. A61K 31/506; A61K 31/519; A61P 35/00

(52) U.S. Cl. CPC A61K 31/506; A61K 31/519; A61P 35/00

(57) ABSTRACT

The specification relates to epidermal growth factor receptor (EGFR) tyrosine kinase inhibitors (TKIs) for use in the treatment of cancer, wherein the EGFR TKI is administered in combination with an AKT inhibitor.

### FIELD

The specification relates to an Epidermal Growth Factor Receptor (EGFR) Tyrosine Kinase Inhibitor (TKI) for use in the treatment of cancer (for example non-small cell lung cancer [NSCLC]), wherein the EGFR TKI is administered in combination with an AKT inhibitor.

### BACKGROUND

The discovery of activating mutations in the epidermal growth factor receptor (EGFR) has revolutionized the treatment of the disease. In 2004 it was reported that activating mutations in exons 18-21 of EGFR correlated with a response to EGFR-TKI therapy in NSCLC (Science [2004], vol. 304, 1497-1500; New England Journal of Medicine [2004], vol. 350, 2129-2139). It is estimated that these mutations are prevalent in approximately 10-16% of NSCLC human patients in the United States and Europe, and in approximately 30-50% of NSCLC human patients in Asia. Two of the most significant EGFR activating mutations are exon 19 deletions and missense mutations in exon 21. Exon 19 deletions account for approximately 45% of known EGFR mutations. Eleven different mutations, resulting in deletion of three to seven amino acids, have been detected in exon 19, all centred around the uniformly deleted codons for amino acids 747-749. The most significant exon 19 deletion is E746-A750. The missense mutations in exon 21 account for approximately 39-45% of known EGFR mutations, of which the substitution mutation L858R accounts for approximately 39% of the total mutations in exon 21 (J. Thorac. Oncol. [2010], 1551-1558).

Two first generation (erlotinib & gefitinib), two second generation (afatinib & dacomitinib) and a third generation (osimertinib) epidermal growth factor receptor (EGFR) tyrosine kinase inhibitors (TKIs) are currently available for the management of EGFR mutation-positive NSCLC. All these TKIs are effective in patients with NSCLC whose tumours harbour the in-frame deletions in exon 19 and the L858R point mutation in exon 21. These two mutations represent approximatively 90% of all EGFR mutations. In approximately 50% of patients, resistance to first- and second-generation EGFR TKI is mediated by the acquisition of the ‘gatekeeper’ mutation T790M. Currently, osimertinib is the only registered EGFR TKI that is active against exon 19 deletions and L858R mutation, regardless of the presence of T790M mutation. However, even patients treated with osimertinib ultimately progress, predominantly due to the development of acquired resistance resulting from other resistance mechanisms. As such, there remains a need to develop new therapies for the treatment of NSCLC, especially for patients whose disease has progressed following treatment with a third generation EGFR TKI.

Induction of programmed cell death via apoptosis is a critical mechanism of the anticancer effects of osimertinib and other EGFR TKIs. However, certain cancers can develop (or intrinsically possess) resistance to such apoptosis, reducing the effectiveness of treatment.

Through laboratory experiments with populations of cancer cells sensitive to osimertinib, it has been found that the effects of EGFR TKIs may be enhanced in some patients by the combined use of AKT inhibitors. AKT is a serine/threonine-specific protein kinase that plays a key role in multiple cellular processes such as glucose metabolism, apoptosis, cell proliferation, transcription, and cell migration. Mammalian cells express three closely related AKT isoforms that are encoded by different genes: AKT1 (protein kinase Bα), AKT2 (protein kinase Bβ), and AKT3 (protein kinase Bγ). Example AKT inhibitors include capivasertib, or a pharmaceutically acceptable salt thereof, (also known as AZD5363 and by the chemical name of (S)-4-amino-N-(1-(4-chlorophenyl)-3-hydroxypropyl)-1-(7H-pyrrolo[2,3-d]pyrimidin-4-yl)piperidine-4-carboxamide), which is a selective inhibitor of all three AKT isoforms.

Without being bound by theory, it is proposed that, in cancer cells reliant on the EGFR pathway, inhibition of this protein may induce a state in which cells are susceptible to AKT inhibitors. Cells that survive chronic treatment with EGFR TKI monotherapy may have a defect in cell death and can act as a reservoir for the development of clinical resistance. However, in a subset of these patients, cellular adaptations required by cancer cells to avoid death in the presence of EGFR inhibition may uncover a novel vulnerability to AKT inhibitors.

In preclinical cell line models, a subset of cells tolerant to osimertinib showed enhanced sensitivity to AKT inhibitors compared to osimertinib-sensitive parental cells. This allowed a combination of AKT and osimertinib to overcome developed resistance, providing a potential pathway to treat patients whose cancer no longer responds to EGFR TKIs alone.

It has therefore been determined that treatment with an AKT inhibitor may overcome such resistance, re-sensitizing cancer to the apoptotic effects of EGFR TKIs. Furthermore, combinations of an EGFR inhibitor and an AKT inhibitor may act together in therapy to prevent resistance or delay its onset.

### SUMMARY

The present specification provides a means for enhancing the anti-proliferative and pro-apoptotic effects of EGFR TKI treatment in cancers (for example NSCLC) utilising AKT inhibitors in combination with EGFR TKIs.

This specification thus discloses a combination of an EGFR TKI and an AKT inhibitor both as a first-line treatment (i.e. in EGFR TKI-naïve patients) and as a treatment at the stage of minimal residual disease (i.e. in patients previously treated with EGFR TKIs, where combination treatment is initiated at the point of maximal drug response, wherein the number of remaining tumour cells may be so small that they do not cause any physical signs or symptoms) of EGFR-mutant NSCLC.

In an aspect, there is provided an EGFR TKI for use in the treatment of cancer in a human patient, wherein the EGFR TKI is administered in combination with an AKT inhibitor.

The terms “treat,” “treating,” and “treatment” refer to at least partially alleviating, inhibiting, preventing and/or ameliorating a condition, disorder, or disease, such as lung cancer. The term “treatment of cancer” includes both in vitro and in vivo treatments, including in warm-blooded animals such as humans. The effectiveness of treatment of cancer can be assessed in a variety of ways, including but not limited to: inhibiting cancer cell proliferation (including the reversal of cancer growth); promoting cancer cell death (e.g., by promoting apoptosis or another cell death mechanism); improvement in symptoms; duration of response to the treatment; delay in progression of disease; and prolonging survival. Treatments can also be assessed with regard to the nature and extent of side effects associated with the treatment. Furthermore, effectiveness can be assessed with regard to biomarkers, such as levels of expression or phosphorylation of proteins known to be associated with particular biological phenomena. Other assessments of effectiveness are known to those of skill in the art.

The phrase “in combination with” similar terms (including “simultaneous”) encompass administration of two or more active pharmaceutical ingredients to a subject and include simultaneous administration in separate compositions, administration at different times in separate compositions, or administration in a composition in which two or more active pharmaceutical ingredients are present.

In a further aspect, there is provided the use of an EGFR TKI in the manufacture of a medicament for the treatment of cancer in a human patient, wherein the EGFR TKI is administered in combination with an AKT inhibitor.

In a further aspect, there is provided a method of treating cancer in a human patient in need of such a treatment, comprising administering to the human patient a therapeutically effective amount of an EGFR TKI, wherein the EGFR TKI is administered in combination with a therapeutically effective amount of an AKT inhibitor.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g. the amount of apoptosis). The specific dose will vary depending on the particular compounds chosen, the dosing regimen to be followed, whether the compound is administered in combination with other compounds, timing of administration, the tissue to which it is administered, and the physical delivery system in which the compound is carried.

In a further aspect, there is provided a method of treating cancer in a human patient in need of such a treatment, comprising administering to the human patient a first amount of an EGFR TKI, and a second amount of an AKT inhibitor, where the first amount and the second amount together comprise a therapeutically effective amount.

In a further aspect, there is provided a pharmaceutical composition comprising an EGFR TKI, an AKT inhibitor and a pharmaceutically acceptable excipient.

The term “pharmaceutically acceptable” is used to specify that an object (for example a salt, dosage form or excipient [such as a diluent or carrier]) is suitable for use in patients. An example list of pharmaceutically acceptable salts can be found in the “Handbook of Pharmaceutical Salts: Properties, Selection and Use”, P. H. Stahl and C. G. Wermuth, editors, Weinheim/Zurich: Wiley-VCH/VFiCA, 2002 or subsequent editions. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminium. Organic bases from which salts can be derived include, for example, primary, secondary, and tertiary amines, substituted amines including naturally occurring substituted amines, cyclic amines and basic ion exchange resins. Examples include isopropylamine, trimethylamine, diethylamine, triethylamine, tripropylamine, and ethanolamine.

In a further aspect, there is provided an AKT inhibitor for use in the treatment of cancer in a human patient, wherein the AKT inhibitor is administered in combination with an EGFR TKI.

### DESCRIPTION OF FIGURES

FIG. 1: Viability assay (CTG) in PC9 PIK3CA^{H1047R} and PC9 PIK3CA^{E453K} cells compared to parental PC9 cells. PC9 PIK3CAm cells are more resistant to osimertinib (A).

FIG. 2: Viability assay (CTG) in PC9 PIK3CA^{H1047R} cells compared to parental PC9 cells. PC9 PIK3CAm cells are more resistant to osimertinib and can be partially re-sensitized with co-treatment with 500 nM AZD5363.

FIG. 3: Viability assay (CTG) in PC9 PIK3CA^{E453K} cells compared to parental PC9 cells. PC9 PIK3CAm cells are more resistant to osimertinib and can be partially re-sensitized with co-treatment with 500 nM AZD5363.

FIG. 4: Osimertinib-mediated apoptotic response in PC9 PIK3CA^{H1047R} cells compared to PC9 parental cells by caspase 3/7 activation assay. Diminished apoptotic response in PC9 PIK3CA^{H1047R} that cannot be restored by co-treatment with AZD5363.

FIG. 5: Clonogenic assay in PC9 PIK3CAm compared to parental PC9 cells. PC9-PIK3CAm cells developed resistance to osimertinib that can be partially rescued by combination with capivasertib.

FIG. 6: WB analysis in PC9 PIK3CAm cells treated with 160 nM osimertinib alone or in combination with capivasertib for 4 h.

FIG. 7: Cell viability (CTG) in two different HCC-827 PTEN^{KO} cell lines compared to parental cells. HCC-827 PTEN^{KO} cells are more resistant to osimertinib compared to parental cells.

FIG. 8: Cell viability (CTG) in naïve or exposed for 3 weeks to osimertinib PC9 PTEN^{KO} cell lines compared to parental cells. PC9 PTEN^{KO} develop resistance after 3 weeks of drug selection.

FIG. 9: Clonogenic assay in PC9 PTEN^{KO} cells treated for 3 or 5 weeks respectively with osimertinib 160 nM alone or in combination with 500 nM AZD5363. PTEN^{KO} cells develop resistance that can be partially re-sensitized by combination with capivasertib.

FIG. 10: Clonogenic assay in HCC-827 PTEN^{KO} cells treated for 3 or 5 weeks respectively with osimertinib 160 nM alone or in combination with 500 nM AZD5363. PTEN^{KO} cells develop resistance that can be partially re-sensitized by combination with capivasertib.

FIG. 11: Intracellular changes by WB analysis in HCC-827 PTEN^{KO} cell lines compared to parental cells.

FIG. 12: Intracellular changes by WB analysis in PC9 PTEN^{KO} (naïve or exposed for 3 weeks to 160 nM osimertinib) cell lines compared to parental cells.

FIG. 13: Intracellular changes by WB analysis in HCC-827 PTEN^{KO} cell line treated with capivasertib as a monotherapy.

FIG. 14: Intracellular changes by WB analysis in HCC-827 PTEN^{KO} cell line treated with capivasertib as a combination with osimertinib therapy.

FIG. 15: Cell viability assay (CTG) in HCC-827 cells treated with osimertinib (3 nM-10 mM) alone or in combination with 500 nM capivasertib.

FIG. 16: Clonogenic assay in 2 different PC9 PTEN^{KO} treated with osimertinib alone or in combination with capivasertib or PI3K inhibitors compared to parental NTC cells (upper panel). PC9 PTEN^{KO} cells develop resistance to osimertinib that can be partially re-sensitised by capivasertib.

FIG. 17: Combination effects using an EGFR TKI and AKT inhibitor in a PC9 PIKC3A^{H1047} xenograft model.

FIG. 18: Combination effects using an EGFR TKI and AKT inhibitor in a PC9 PIKC3A^{E453K} xenograft model.

FIG. 19: Combination effects using an EGFR TKI and AKT inhibitor in an LC-F-12 PIKC3A^{E545K} xenograft model.

FIG. 20: Combination effects using an EGFR TKI and AKT inhibitor in an MR131 PTEN^{D326H} xenograft model.

FIG. 21: Combination effects using an EGFR TKI and AKT inhibitor in a CTG-2939 PTEN^{DeepDel} xenograft model.

FIG. 22: Combination effects using an EGFR TKI and AKT inhibitor in a CTG-2180 PTEN^{C304fs} xenograft model.

FIG. 23: Combination effects using an EGFR TKI and AKT inhibitor in a PC9 PTEN^{KO} xenograft model.

FIG. 24: Combination effects using an EGFR TKI and AKT inhibitor in a HCC-827 PTEN^{KO} xenograft model.

### DETAILED DESCRIPTION

### EGFR Mutation Positive NSCLC, Patient Selection and Diagnostic Methods

In embodiments, the cancer comprises a PIK3CA mutation (for example a gain of function mutation, or a deletion, substitution or insertion mutation, such as a PIK3CA^{H1047R} mutation, PIKC3A^{E453K} mutation, PI3K^{E542K} mutation or PIKC3A^{E545K} mutation). PI3KCA mutation status can be determined by methods known in the art. In embodiments, the cancer is PTEN deficient (e.g. comprises a cancerous cell (e.g. a population of cancerous cells, such as the majority of cancerous cells) with a reduction in the normal amount [e.g. compared to a non-cancerous cell of the same patient] or function of the PTEN tumour suppression protein). PTEN status can be determined by methods known in the art.

In embodiments, the cancer is lung cancer, such as non-small cell lung cancer (NSCLC).

In embodiments, the NSCLC is an EGFR mutation-positive NSCLC.

In embodiments, the EGFR mutation-positive NSCLC comprises activating mutations in EGFR. In further embodiments, the EGFR mutation-positive NSCLC comprises non-resistant mutations. In further embodiments, the activating mutations in EGFR comprise activating mutations in exons 18-21. In further embodiments, the activating mutations in EGFR comprise exon 19 deletions or missense mutations in exon 21. In further embodiments, the activating mutations in EGFR comprise exon 19 deletions or L858R substitution mutations. In further embodiments, the mutations in EGFR comprise a T790M mutation.

In embodiments, the EGFR mutation-positive NSCLC is a locally advanced EGFR mutation-positive NSCLC.

In embodiments, the EGFR mutation-positive NSCLC is a metastatic EGFR mutation-positive NSCLC.

In embodiments, the EGFR mutation-positive NSCLC is not amenable to curative surgery or radiotherapy. There are numerous methods to detect EGFR activating mutations, of which the skilled person will be aware. A number of tests suitable for use in these methods have been approved by the US Food and Drug Administration (FDA). These include both tumour tissue and plasma based diagnostic methods. In general, the EGFR mutation status is first assessed using a tumour tissue biopsy sample derived from the human patient. If a tumour sample is unavailable, or if the tumour sample is negative, the EGFR mutation status may be assessed using a plasma sample. A particular example of a suitable diagnostic test to detect EGFR mutations, and in particular to detect exon 19 deletions, L858R substitution mutations and the T790M mutation, is the Cobas™ EGFR Mutation Test v2 (Roche Molecular Diagnostics).

In embodiments, therefore, the EGFR mutation-positive NSCLC comprises activating mutations in EGFR (such as activating mutations in exons 18-21, for example exon 19 deletions, missense mutations in exon 21, and L858R substitution mutations; and resistance mutations such as the T790M mutation), wherein the EGFR mutation status of the human patient has been determined using an appropriate diagnostic test. In further embodiments, the EGFR mutation status has been determined using a tumour tissue sample. In further embodiments, the EGFR mutation status has been determined using a plasma sample. In further embodiments, the diagnostic method uses an FDA-approved test. In further embodiments, the diagnostic method uses the Cobas™ EGFR Mutation Test (v1 or v2).

In embodiments, the human patient is an EGFR TKI-naïve human patient.

In embodiments, the human patient has previously received EGFR TKI treatment. In embodiments the human patient has previously been treated with osimertinib or a pharmaceutically acceptable salt thereof.

In further embodiments, the human patient's disease has reached the stage of maximal response (minimal residual disease) during or after previous EGFR TKI treatment. In further embodiments, the human patient's disease has reached maximal response during or after previous treatment with osimertinib or a pharmaceutically acceptable salt thereof. EGFR TKI treatment includes treatment with either a first-, second- or third-generation EGFR TKI or combinations thereof. In embodiments, the human patient has developed EGFR T790M mutation-positive NSCLC.

### EGFR TKIs

EGFR TKIs can be characterised as either first-, second- or third-generation EGFR TKIs, as set out below.

First-generation EGFR TKIs are reversible inhibitors of EGFR bearing activating mutations that do not significantly inhibit EGFR bearing the T790M mutation. Examples of first-generation TKIs include gefitinib and erlotinib.

Second-generation EGFR TKIs are irreversible inhibitors of EGFR bearing activating mutations that do not significantly inhibit EGFR bearing the T790M mutation. Examples of second-generation TKIs include afatinib and dacomitinib.

Third-generation EGFR TKIs are inhibitors of EGFR bearing activating mutations that also significantly inhibit EGFR bearing the T790M mutation and do not significantly inhibit wild-type EGFR. Examples of third-generation TKIs include compounds of Formula (I), osimertinib, AZD3759 (zorifertinib), lazertinib, nazartinib (EGF816), CO1686 (rociletinib), HM61713 (olmutinib), ASP8273 (naquotinib), PF-06747775 (mavelertinib), avitinib (abivertinib), alflutinib (AST2818), CX-101 (olafertinib; RX-518), aumolertinib (HS-10296; almonertinib) and BPI-7711 (rezivertinib).

In an aspect, the EGFR TKI is a first-generation EGFR TKI. In further embodiments, the first-generation EGFR TKI is selected from the group consisting of gefitinib or a pharmaceutically acceptable salt thereof, icotinib or a pharmaceutically acceptable salt thereof, and erlotinib or a pharmaceutically acceptable salt thereof.

In an aspect, the EGFR TKI is a second-generation EGFR TKI. In a further aspect, the second-generation EGFR TKI is selected from dacomitinib, or a pharmaceutically acceptable salt thereof, and afatinib or a pharmaceutically acceptable salt thereof.

In an aspect, the EGFR TKI is a third-generation EGFR TKI. In a further aspect, the third-generation EGFR TKI is a compound of Formula (I), as defined below. In a further aspect, the third-generation EGFR TKI is selected from the group consisting of osimertinib or a pharmaceutically acceptable salt thereof, AZD3759 or a pharmaceutically acceptable salt thereof, lazertinib or a pharmaceutically acceptable salt thereof, abivertinib or a pharmaceutically acceptable salt thereof, alflutinib or a pharmaceutically acceptable salt thereof, CX-101 or a pharmaceutically acceptable salt thereof, HS-10296 or a pharmaceutically acceptable salt thereof and BPI-7711 or a pharmaceutically acceptable salt thereof. In a further aspect, the third generation EGFR TKI is osimertinib or a pharmaceutically acceptable salt thereof.

### Compounds of Formula (I)

In an aspect, the EGFR TKI is a compound of Formula (I):

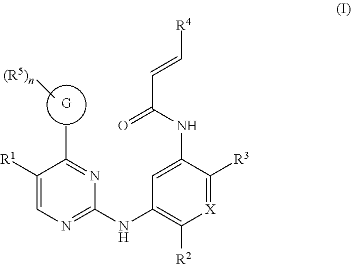

wherein:
G is selected from 4,5,6,7-tetrahydropyrazolo[1,5-a]pyridin-3-yl, indol-3-yl, indazol-1-yl, 3,4-dihydro-1H-[1,4]oxazino[4,3-a]indol-10-yl, 6,7,8,9-tetrahydropyrido[1,2-a]indol-10-yl, 5,6-dihydro-4H-pyrrolo[3,2,1-ij]quinolin-1-yl, pyrrolo[3,2-b]pyridin-3-yl and pyrazolo[1,5-α]pyridin-3-yl;
R¹ is selected from hydrogen, fluoro, chloro, methyl and cyano;
R² is selected from methoxy, trifluoromethoxy, ethoxy, 2,2,2-trifluoroethoxy and methyl;
R³ is selected from (3R)-3-(dimethylamino)pyrrolidin-1-yl, (3S)-3-(dimethyl-amino)pyrrolidin-1-yl, 3-(dimethylamino)azetidin-1-yl, [2-(dimethylamino)ethyl]-(methyl)amino, [2-(methylamino)ethyl](methyl)amino, 2-(dimethylamino)ethoxy, 2-(methylamino)ethoxy, 5-methyl-2,5-diazaspiro[3.4]oct-2-yl, (3aR,6aR)-5-methylhexa-hydro-pyrrolo[3,4-b]pyrrol-1 (2H)-yl, 1-methyl-1,2,3,6-tetrahydropyridin-4-yl, 4-methylpiperizin-1-yl, 4-[2-(dimethylamino)-2-oxoethyl]piperazin-1-yl, methyl[2-(4-methylpiperazin-1-yl)ethyl]amino, methyl[2-(morpholin-4-yl)ethyl]amino, 1-amino-1,2,3,6-tetrahydropyridin-4-yl and 4-[(2S)-2-aminopropanoyl]piperazin-1-yl;
R⁴ is selected from hydrogen, 1-piperidinomethyl and N,N-dimethylaminomethyl;
R⁵ is independently selected from methyl, ethyl, propyl, 2,2-difluoroethyl, 2,2,2-trifluoroethyl, fluoro, chloro and cyclopropyl;
X is CH or N; and
n is 0, 1 or 2;
or a pharmaceutically acceptable salt thereof.

In a further aspect there is provided a compound of Formula (I), as defined above, wherein G is selected from indol-3-yl and indazol-1-yl; R¹ is selected from hydrogen, fluoro, chloro, methyl and cyano; R² is selected from methoxy and 2,2,2-trifluoroethoxy; R³ is selected from [2-(dimethylamino)ethyl]-(methyl)amino, [2-(methylamino)ethyl](methyl)amino, 2-(dimethylamino)ethoxy and 2-(methylamino)ethoxy; R⁴ is hydrogen; R⁵ is selected from methyl, 2,2,2-trifluoroethyl and cyclopropyl; X is CH or N; and n is 0 or 1; or a pharmaceutically acceptable salt thereof.

Examples of compounds of Formula (I) include those described in WO 2013/014448, WO 2015/175632, WO 2016/054987, WO 2016/015453, WO 2016/094821, WO 2016/070816 and WO 2016/173438.

### Osimertinib and Pharmaceutical Compositions Thereof

Osimertinib has the following chemical structure:

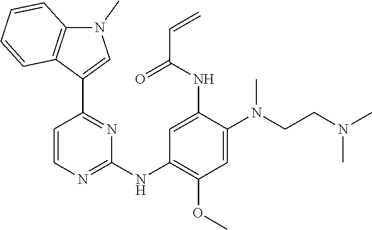 [SMILES: C=CC(=O)Nc1cc(Nc2nccc(-c3cn(C)c4ccccc34)n2)c(OC)cc1N(C)CCN(C)C]

The free base of osimertinib is known by the chemical name: N-(2-{2-dimethylamino ethyl-methylamino}-4-methoxy-5-{[4-(1-methylindol-3-yl)pyrimidin-2-yl]amino}phenyl)prop-2-enamide. Osimertinib is described in WO 2013/014448. Osimertinib is also known as AZD9291.

Osimertinib may be found in the form of the mesylate salt: N-(2-{2-dimethylamino ethyl-methylamino}-4-methoxy-5-{[4-(1-methylindol-3-yl)pyrimidin-2-yl]amino}phenyl)prop-2-enamide mesylate salt. Osimertinib mesylate is also known as TAGRISSO™.

Osimertinib mesylate is currently approved as an oral once daily tablet formulation, at a dose of 80 mg (expressed as free base, equivalent to 95.4 mg osimertinib mesylate), for the treatment of metastatic EGFR T790M mutation positive NSCLC patients. A 40 mg oral once daily tablet formulation (expressed as free base, equivalent to 47.7 mg osimertinib mesylate) is available should dose modification be required. The tablet core comprises pharmaceutical diluents (such as mannitol and microcrystalline cellulose), disintegrants (such as low-substituted hydroxypropyl cellulose) and lubricants (such as sodium stearyl fumarate). The tablet formulation is described in WO 2015/101791.

In an aspect, therefore, osimertinib, or a pharmaceutically acceptable salt thereof, is in the form of the mesylate salt, i.e. N-(2-{2-dimethylamino ethyl-methylamino}-4-methoxy-5-{[4-(1-methylindol-3-yl)pyrimidin-2-yl]amino}phenyl)prop-2-enamide mesylate salt.

In an aspect, osimertinib, or a pharmaceutically acceptable salt thereof, is administered once-daily. In a further aspect, osimertinib mesylate is administered once-daily.

In an aspect, the total daily dose of osimertinib is about 80 mg. In a further aspect, the total daily dose of osimertinib mesylate is about 95.4 mg.

In an aspect, the total daily dose of osimertinib is about 40 mg. In a further aspect, the total daily dose of osimertinib mesylate is about 47.7 mg.

In an aspect, osimertinib, or a pharmaceutically acceptable salt thereof, is in tablet form.

In an aspect, osimertinib, or a pharmaceutically acceptable salt thereof, is administered in the form of a pharmaceutical composition comprising one or more pharmaceutically acceptable excipients (for example a diluent or carrier). In a further aspect, the composition comprises one or more pharmaceutical diluents (such as mannitol and microcrystalline cellulose), one or more pharmaceutical disintegrants (such as low-substituted hydroxypropyl cellulose) or one or more pharmaceutical lubricants (such as sodium stearyl fumarate).

In an aspect, the composition is in the form of a tablet, wherein the tablet core comprises: (a) from 2 to 70 parts of osimertinib, or a pharmaceutically acceptable salt thereof; (b) from 5 to 96 parts of two or more pharmaceutical diluents; (c) from 2 to 15 parts of one or more pharmaceutical disintegrants; and (d) from 0.5 to 3 parts of one or more pharmaceutical lubricants; and wherein all parts are by weight and the sum of the parts (a)+(b)+(c)+(d)=100.

In an aspect, the composition is in the form of a tablet, wherein the tablet core comprises: (a) from 7 to 25 parts of osimertinib, or a pharmaceutically acceptable salt thereof; (b) from 55 to 85 parts of two or more pharmaceutical diluents, wherein the pharmaceutical diluents comprise microcrystalline cellulose and mannitol; (c) from 2 to 8 parts of pharmaceutical disintegrant, wherein the pharmaceutical disintegrant comprises low-substituted hydroxypropyl cellulose; (d) from 1.5 to 2.5 parts of pharmaceutical lubricant, wherein the pharmaceutical lubricant comprises sodium stearyl fumarate; and wherein all parts are by weight and the sum of the parts (a)+(b)+(c)+(d)=100.

In an aspect, the composition is in the form of a tablet, wherein the tablet core comprises: (a) about 19 parts of osimertinib mesylate; (b) about 59 parts of mannitol; (c) about 15 parts of microcrystalline cellulose; (d) about 5 parts of low-substituted hydroxypropyl cellulose; and (e) about 2 parts of sodium stearyl fumarate; and wherein all parts are by weight and the sum of the parts (a)+(b)+(c)+(d)+(e)=100.

### AZD3759 (Zorifertinib)

AZD3759 has the following chemical structure:

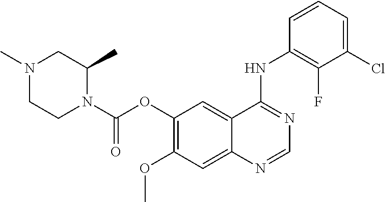 [SMILES: COc1cc2ncnc(Nc3cccc(Cl)c3F)c2cc1OC(=O)N1CCN(C)C[C@H]1C]

The free base of AZD3759 is known by the chemical name: 4-[(3-chloro-2-fluorophenyl)amino]-7-methoxy-6-quinazolinyl (2R)-2,4-dimethyl-1-piperazinecarboxylate. AZD3759 is described in WO 2014/135876.

In an aspect, AZD3759, or a pharmaceutically acceptable salt thereof, is administered twice-daily. In a further aspect, AZD3759 is administered twice-daily.

In an aspect, the total daily dose of AZD3759 is about 400 mg. In a further aspect, about 200 mg of AZD3759 is administered twice a day.

### Lazertinib

Lazertinib has the following chemical structure:

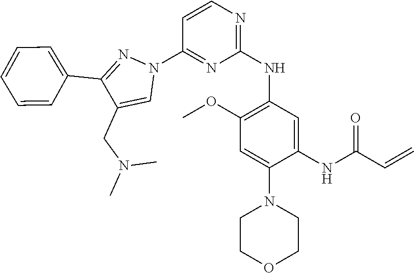 [SMILES: C=CC(=O)Nc1cc(Nc2nccc(-n3cc(CN(C)C)c(-c4ccccc4)n3)n2)c(OC)cc1N1CCOCC1]

The free base of lazertinib is known by the chemical name N-{5-[(4-{4-[(dimethylamino)methyl]-3-phenyl-1H-pyrazol-1-yl}-2-pyrimidinyl)amino]-4-methoxy-2-(4-morpholinyl)phenyl}acrylamide. Lazertinib is described in WO 2016/060443. Lazertinib is also known by the names YH25448 and GNS-1480.

In an aspect, lazertinib, or a pharmaceutically acceptable salt thereof, is administered once-daily. In a further aspect, lazertinib is administered once-daily.

In an aspect, the total daily dose of lazertinib is about 20 to 320 mg.

In an aspect, the total daily dose of lazertinib is about 240 mg.

### Avitinib

Avitinib has the following chemical structure:

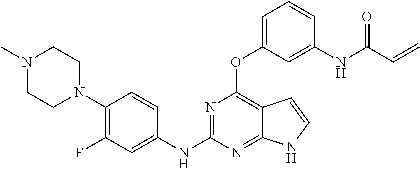 [SMILES: C=CC(=O)Nc1cccc(Oc2nc(Nc3ccc(N4CCN(C)CC4)c(F)c3)nc3[nH]ccc23)c1]

The free base of avitinib is known by the chemical name: N-(3-((2-((3-fluoro-4-(4-methylpiperazin-1-yl)phenyl)amino)-7H-pyrrolo (2,3-d)pyrimidin-4-yl)oxy)phenyl)prop-2-enamide. Avitinib is disclosed in US2014038940. Avitinib is also known as abivertinib.

In an aspect, avitinib or a pharmaceutically acceptable salt thereof, is administered twice daily. In a further aspect, avitinib maleate is administered twice daily.

In an aspect, the total daily dose of avitinib maleate is about 600 mg.

### Alflutinib

Alflutinib has the following chemical structure:

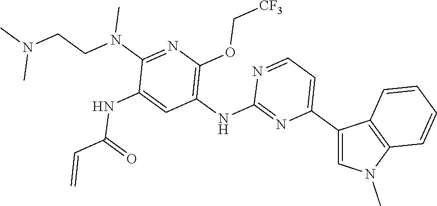 [SMILES: C=CC(=O)Nc1cc(Nc2nccc(-c3cn(C)c4ccccc34)n2)c(OCC(F)(F)F)nc1N(C)CCN(C)C]

The free base of alflutinib is known by the chemical name: N-{2-{[2-(dimethylamino)ethyl](methyl)amino}-6-(2,2,2-trifluoroethoxyl)-5-{[4-(1-methyl-1H-indol-3-yl)pyrimidin-2-yl]amino}pyridin-3-yl}acrylamide. Alflutinib is disclosed in WO 2016/15453. Alflutinib is also known as AST2818.

In an aspect, alflutinib or a pharmaceutically acceptable salt thereof, is administered once daily. In a further aspect, alflutinib mesylate is administered once daily.

In an aspect, the total daily dose of alflutinib mesylate is about 80 mg.

In an aspect, the total daily dose of alflutinib mesylate is about 40 mg.

### Afatinib

Afatinib has the following chemical structure:

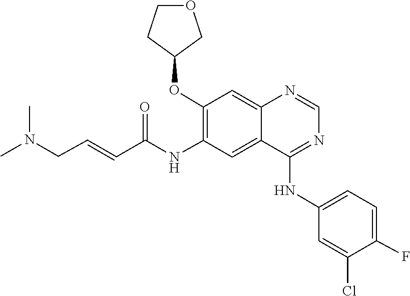

The free base of afatinib is known by the chemical name: N-[4-(3-chloro-4-fluoroanilino)-7-[(3S)-oxolan-3-yl] oxyquinazolin-6-yl]-4-(dimethylamino)but-2-enamide. Afatinib is disclosed in WO 02/50043. Afatinib is also known as Gilotrif.

In an aspect, afatinib or a pharmaceutically acceptable salt thereof, is administered once daily. In a further aspect, afatinib dimaleate is administered once daily.

In an aspect, the total daily dose of afatinib dimaleate is about 40 mg.

In an aspect, the total daily dose of afatinib dimaleate is about 30 mg.

### CX-101

CX-101 has the following chemical structure:

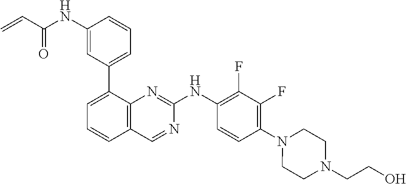 [SMILES: C=CC(=O)Nc1cccc(-c2cccc3cnc(Nc4ccc(N5CCN(CCO)CC5)c(F)c4F)nc23)c1]

The free base of CX-101 is known by the chemical name: N-(3-(2-((2,3-difluoro-4-(4-(2-hydroxyethyl)piperazin-1-yl)phenyl)amino) quinazolin-8-yl)phenyl) acrylamide. CX-101 is disclosed in WO 2015/027222. CX-101 is also known as RX-518 and olafertinib.

### HS-10296 (Almonertinib; Aumolertinib)

HS-10296 (almonertinib; aumolertinib) has the following chemical structure:

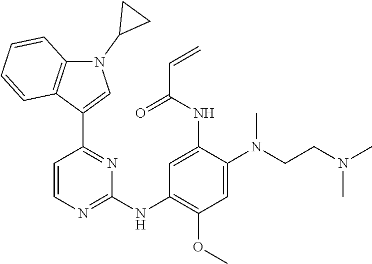 [SMILES: C=CC(=O)Nc1cc(Nc2nccc(-c3cn(C4CC4)c4ccccc34)n2)c(OC)cc1N(C)CCN(C)C]

The free base of HS-10296 is known by the chemical name: N-[5-[[4-(1-cyclopropylindol-3-yl)pyrimidin-2-yl]amino]-2-[2-(dimethylamino)ethyl-methyl-amino]-4-methoxy-phenyl]prop-2-enamide. HS-10296 is disclosed in WO 2016/054987.

In an aspect, the total daily dose of HS-10296 is about 110 mg.

### BPI-7711 (Rezivertinib)

BPI-7711 has the following chemical structure:

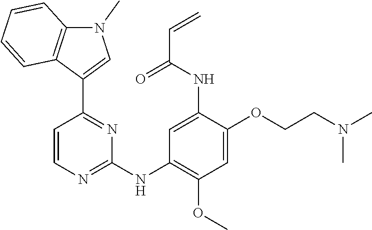 [SMILES: C=CC(=O)Nc1cc(Nc2nccc(-c3cn(C)c4ccccc34)n2)c(OC)cc1OCCN(C)C]

The free base of BPI-7711 is known by the chemical name: N-[2-[2-(dimethylamino)ethoxy]-4-methoxy-5-[[4-(1-methylindol-3-yl)pyrimidin-2-yl]amino]phenyl]prop-2-enamide. BPI-7711 is disclosed in WO 2016/94821.

In an aspect, the total daily dose of BPI-7711 is about 180 mg.

### Dacomitinib

Dacomitinib has the following chemical structure:

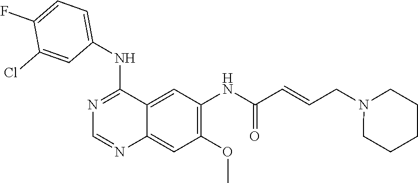 [SMILES: COc1cc2ncnc(Nc3ccc(F)c(Cl)c3)c2cc1NC(=O)/C=C/CN1CCCCC1]

The free form of dacomitinib is known by the chemical name: (2E)-N-{4-[(3-chloro-4-fluorophenyl)amino]-7-methoxyquinazolin-6-yl}-4-(piperidin-1-yl)but-2-enamide. Dacomitinib is described in WO 2005/107758. Dacomitinib is also known by the name PF-00299804.

Dacomitinib may be found in the form of dacomitinib monohydrate, i.e. (2E)-N-{4-[(3-chloro-4-fluorophenyl)amino]-7-methoxyquinazolin-6-yl}-4-(piperidin-1-yl)but-2-enamide monohydrate.

In an aspect, dacomitinib, or a pharmaceutically acceptable salt thereof, is administered once-daily. In a further aspect, dacomitinib monohydrate is administered once-daily.

In an aspect, the total daily dose of dacomitinib monohydrate is about 45 mg.

In an aspect, dacomitinib, or a pharmaceutically acceptable salt thereof, is in tablet form.

In an aspect, dacomitinib, or a pharmaceutically acceptable salt thereof, is administered in the form of a pharmaceutical composition comprising one or more pharmaceutically acceptable excipients. In a further aspect, the one or more pharmaceutically acceptable excipients comprise lactose monohydrate, microcrystalline cellulose, sodium starch glycolate and magnesium stearate.

### Icotinib

Icotinib has the following chemical structure:

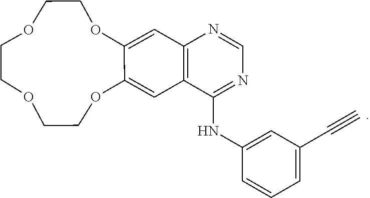 [SMILES: C#Cc1cccc(Nc2ncnc3cc4c(cc23)OCCOCCOCCO4)c1]

The free base of icotinib is known by the chemical name: N-(3-ethynylphenyl)-2,5,8,11-tetraoxa-15,17-diazatricyclo[10.8.0.0^{14,19}]icosa-1(12),13,15,17,19-pentaen-18-amine. Icotinib is disclosed in WO2013064128. Icotinib is also known as Conmana.

In embodiments, icotinib, or a pharmaceutically acceptable salt thereof, is administered three times daily.

In further embodiments, icotinib hydrochloride is administered three times daily.

In embodiments, the total daily dose of icotinib hydrochloride is about 375 mg.

### Gefitinib

Gefitinib has the following chemical structure:

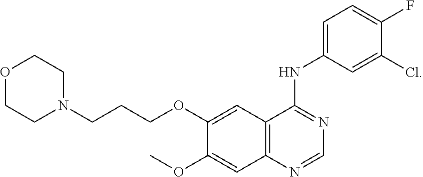 [SMILES: COc1cc2ncnc(Nc3ccc(F)c(Cl)c3)c2cc1OCCCN1CCOCC1]

The free base of gefitinib is known by the chemical name: N-(3-chloro-4-fluorophenyl)-7-methoxy-6-(3-morpholin-4-ylpropoxy) quinazolin-4-amine. Gefitinib is disclosed in WO 1996/033980. Gefitinib is also known as IRESSA™.

In embodiments, gefitinib, or a pharmaceutically acceptable salt thereof, is administered once-daily. In further embodiments, gefitinib is administered once-daily.

In embodiments, the total daily dose of gefitinib is about 250 mg.

### Erlotinib

Erlotinib has the following chemical structure:

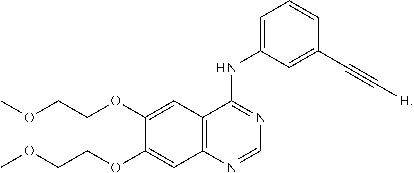

The free base of erlotinib is known by the chemical name: N-(3-ethynylphenyl)-6,7-bis(2-methoxyethoxy) quinazolin-4-amine. Erlotinib is disclosed in WO 1996/030347. Erlotinib is also known as TARCEVA™.

In embodiments, erlotinib, or a pharmaceutically acceptable salt thereof, is administered once-daily. In further embodiments, erlotinib is administered once-daily.

In embodiments, the total daily dose of erlotinib is about 150 mg.

In embodiments, the total daily dose of erlotinib is about 100 mg.

### AKT Inhibitors

In embodiments, the AKT inhibitor is any molecule which binds to and inhibits the activity of one or more AKT isoforms.

In embodiments, the AKT inhibitor is selected from the group consisting of miransertib (ARQ-092) or a pharmaceutically acceptable salt thereof, BAY1125976 or a pharmaceutically acceptable salt thereof, borussertib or a pharmaceutically acceptable salt thereof, AT7867 or a pharmaceutically acceptable salt thereof, CCT128930 or a pharmaceutically acceptable salt thereof, A-674563 or a pharmaceutically acceptable salt thereof, PHT-427 or a pharmaceutically acceptable salt thereof, Akti-1/2 or a pharmaceutically acceptable salt thereof, AT13148 or a pharmaceutically acceptable salt thereof, SC79 or a pharmaceutically acceptable salt thereof, capivasertib or a pharmaceutically acceptable salt thereof, miltefosine or a pharmaceutically acceptable salt thereof, perifosine or a pharmaceutically acceptable salt thereof, MK-2206 or a pharmaceutically acceptable salt thereof, RX-0201 or a pharmaceutically acceptable salt thereof, erucylphosphocholine or a pharmaceutically acceptable salt thereof, PBI-05204 or a pharmaceutically acceptable salt thereof, GSK690693 or a pharmaceutically acceptable salt thereof, afuresertib (GSK2110183) or a pharmaceutically acceptable salt thereof, uprosertib (GSK2141795) or a pharmaceutically acceptable salt thereof, XL-418 or a pharmaceutically acceptable salt thereof and ipatasertib (GDC-0068) or a pharmaceutically acceptable salt thereof.

In embodiments, the AKT inhibitor is selected from the group consisting of capivasertib or a pharmaceutically acceptable salt thereof, perifosine or a pharmaceutically acceptable salt thereof, MK-2206 or a pharmaceutically acceptable salt thereof, RX-0201 or a pharmaceutically acceptable salt thereof, erucylphosphocholine or a pharmaceutically acceptable salt thereof, PBI-05204 or a pharmaceutically acceptable salt thereof, GSK690693 or a pharmaceutically acceptable salt thereof, uprosertib (GSK2141795) or a pharmaceutically acceptable salt thereof, XL-418 or a pharmaceutically acceptable salt thereof and ipatasertib or a pharmaceutically acceptable salt thereof.

In embodiments, the AKT inhibitor is selected from the group consisting of capivasertib or a pharmaceutically acceptable salt thereof, perifosine or a pharmaceutically acceptable salt thereof, MK-2206 or a pharmaceutically acceptable salt thereof, GSK690693 or a pharmaceutically acceptable salt thereof, afuresertib (GSK2110183) or a pharmaceutically acceptable salt thereof, uprosertib (GSK2141795) or a pharmaceutically acceptable salt thereof and ipatasertib (GDC-0068) or a pharmaceutically acceptable salt thereof.

### Capivasertib

Capivasertib has the following chemical structure:

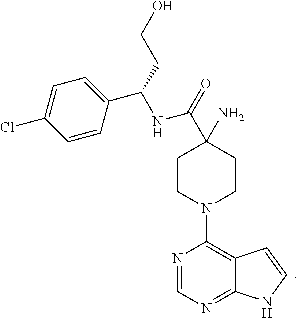 [SMILES: NC1(C(=O)N[C@@H](CCO)c2ccc(Cl)cc2)CCN(c2ncnc3[nH]ccc23)CC1]

The free base of capivasertib is known by the chemical name(S)-4-amino-N-(1-(4-chlorophenyl)-3-hydroxypropyl)-1-(7H-pyrrolo[2,3-d]pyrimidin-4-yl)piperidine-4-carboxamide). Capivasertib is disclosed in WO2009/047563, which discloses capivasertib (in Example 9) and describes its synthesis.

In an aspect, capivasertib, or a pharmaceutically acceptable salt thereof, is administered in the form of a pharmaceutical composition comprising one or more pharmaceutically acceptable excipients. In a further aspect, the composition comprises one or more pharmaceutical diluents (such as mannitol and microcrystalline cellulose), one or more pharmaceutical disintegrants (such as low-substituted hydroxypropyl cellulose) or one or more pharmaceutical lubricants (such as sodium stearyl fumarate).

In an aspect, the composition is in the form of a tablet.

In combinations with osimertinib, capivasertib, or a pharmaceutically acceptable salt thereof, generally is administered to the subject at a daily dosage from about 100 mg to about 1600 mg.

In some embodiments, capivasertib, or a pharmaceutically acceptable salt thereof, is administered at a daily dosage from about 150 mg to about 1500 mg. In one aspect, capivasertib, or a pharmaceutically acceptable salt thereof, is administered at a daily dosage from about 200 mg to about 1400 mg. In another aspect, capivasertib, or a pharmaceutically acceptable salt thereof, is administered at a daily dosage from about 300 mg to about 1300 mg. In another aspect, capivasertib, or a pharmaceutically acceptable salt thereof, is administered at a daily dosage from about 400 mg to about 1200 mg. In another aspect, capivasertib, or a pharmaceutically acceptable salt thereof, is administered at a daily dosage from about 500 mg to about 1100 mg. In another aspect, capivasertib, or a pharmaceutically acceptable salt thereof, is administered at a daily dosage from about 600 mg to about 1000 mg. In some embodiments, capivasertib, or a pharmaceutically acceptable salt thereof, is administered to the subject once daily (QD).

In one aspect, capivasertib, or a pharmaceutically acceptable salt thereof, is administered at a dosage from about 100 mg to about 1000 mg once daily.

In another aspect, capivasertib, or a pharmaceutically acceptable salt thereof, is administered at a dosage from about 150 mg to about 900 mg once daily. In another aspect, capivasertib, or a pharmaceutically acceptable salt thereof, is administered at a dosage from about 200 mg to about 850 mg once daily. In another aspect, capivasertib, or a pharmaceutically acceptable salt thereof, is administered at a dosage from about 250 mg to about 800 mg once daily. In another aspect, capivasertib, or a pharmaceutically acceptable salt thereof, is administered at a dosage from about 300 mg to about 750 mg once daily.

In another aspect, capivasertib, or a pharmaceutically acceptable salt thereof, is administered at a dosage from about 350 mg to about 700 mg once daily. In another aspect, capivasertib, or a pharmaceutically acceptable salt thereof, is administered at a dosage from about 400 mg to about 650 mg once daily.

In some embodiments, capivasertib, or a pharmaceutically acceptable salt thereof, is administered to the subject twice daily (BID). In one aspect, capivasertib, or a pharmaceutically acceptable salt thereof, is administered at a dosage from about 50 mg to about 900 mg twice daily. In another aspect, capivasertib, or a pharmaceutically acceptable salt thereof, is administered at a dosage from about 100 mg to about 875 mg twice daily. In another aspect, capivasertib, or a pharmaceutically acceptable salt thereof, is administered at a dosage from about 200 mg to about 850 mg twice daily. In another aspect, capivasertib, or a pharmaceutically acceptable salt thereof, is administered at a dosage from about 250 mg to about 825 mg twice daily. In another aspect, capivasertib, or a pharmaceutically acceptable salt thereof, is administered at a dosage from about 150 mg to about 250 mg twice daily. In another aspect, capivasertib, or a pharmaceutically acceptable salt thereof, is administered at a dosage from about 250 mg to about 350 mg twice daily. In another aspect, capivasertib, or a pharmaceutically acceptable salt thereof, is administered at a dosage from about 350 mg to about 450 mg twice daily. In another aspect, capivasertib, or a pharmaceutically acceptable salt thereof, is administered at a dosage from about 450 mg to about 550 mg twice daily. In another aspect, capivasertib, or a pharmaceutically acceptable salt thereof, is administered at a dosage from about 550 mg to about 650 mg twice daily. In another aspect, capivasertib, or a pharmaceutically acceptable salt thereof, is administered at a dosage from about 650 mg to about 750 mg twice daily. In another aspect, capivasertib, or a pharmaceutically acceptable salt thereof, is administered at a dosage from about 750 mg to about 850 mg twice daily. In another aspect, capivasertib, or a pharmaceutically acceptable salt thereof, is administered at a dosage of about 160 mg twice daily. In another aspect, capivasertib, or a pharmaceutically acceptable salt thereof, is administered at a dosage of about 200 mg twice daily. In another aspect, capivasertib, or a pharmaceutically acceptable salt thereof, is administered at a dosage of about 240 mg twice daily. In another aspect, capivasertib, or a pharmaceutically acceptable salt thereof, is administered at a dosage of about 280 mg twice daily. In another aspect, capivasertib, or a pharmaceutically acceptable salt thereof, is administered at a dosage of about 320 mg twice daily. In another aspect, capivasertib, or a pharmaceutically acceptable salt thereof, is administered at a dosage of about 360 mg twice daily. In another aspect, capivasertib, or a pharmaceutically acceptable salt thereof, is administered at a dosage of about 400 mg twice daily. In another aspect, capivasertib, or a pharmaceutically acceptable salt thereof, is administered at a dosage of about 440 mg twice daily. In another aspect, capivasertib, or a pharmaceutically acceptable salt thereof, is administered at a dosage of about 480 mg twice daily. In another aspect, capivasertib, or a pharmaceutically acceptable salt thereof, is administered at a dosage of about 520 mg twice daily. In another aspect, capivasertib, or a pharmaceutically acceptable salt thereof, is administered at a dosage of about 560 mg twice daily. In another aspect, capivasertib, or a pharmaceutically acceptable salt thereof, is administered at a dosage of about 600 mg twice daily. In another aspect, capivasertib, or a pharmaceutically acceptable salt thereof, is administered at a dosage of about 640 mg twice daily. In another aspect, capivasertib, or a pharmaceutically acceptable salt thereof, is administered at a dosage of about 680 mg twice daily. In another aspect, capivasertib, or a pharmaceutically acceptable salt thereof, is administered at a dosage of about 720 mg twice daily. In another aspect, capivasertib, or a pharmaceutically acceptable salt thereof, is administered at a dosage of about 760 mg twice daily. In another aspect, capivasertib, or a pharmaceutically acceptable salt thereof, is administered at a dosage of about 800 mg twice daily.

In some embodiments, capivasertib, or a pharmaceutically acceptable salt thereof, is administered under a continuous dosing schedule. In one aspect, for example, capivasertib, or a pharmaceutically acceptable salt thereof, is administered for more than 1, 2, 3, 4, 5, 6, 7, 14, 21, 28, 35, 42, 49, or 56 days. In another aspect, the dosing cycle is 28 days. Administration of capivasertib, or a pharmaceutically acceptable salt thereof, and repeat of the dosing cycle can continue as long as tolerable and beneficial for the subject.

In some embodiments, capivasertib, or a pharmaceutically acceptable salt thereof, is administered once daily (QD) under a continuous dosing schedule. In one aspect, capivasertib, or a pharmaceutically acceptable salt thereof, is administered once daily under a continuous dosing schedule at a dosage from about 100 mg to about 900 mg. In another aspect, capivasertib, or a pharmaceutically acceptable salt thereof, is administered once daily under a continuous dosing schedule at a dosage from about 150 mg to about 875 mg. In another aspect, capivasertib, or a pharmaceutically acceptable salt thereof, is administered once daily under a continuous dosing schedule at a dosage from about 175 mg to about 850 mg. In another aspect, capivasertib, or a pharmaceutically acceptable salt thereof, is administered once daily under a continuous dosing schedule at a dosage from about 200 mg to about 825 mg. In another aspect, capivasertib, or a pharmaceutically acceptable salt thereof, is administered once daily under a continuous dosing schedule at a dosage from about 225 mg to about 800 mg. In another aspect, capivasertib, or a pharmaceutically acceptable salt thereof, is administered once daily under a continuous dosing schedule at a dosage from about 250 mg to about 750 mg. In another aspect, capivasertib, or a pharmaceutically acceptable salt thereof, is administered once daily under a continuous dosing schedule at a dosage from about 275 mg to about 700 mg. In another aspect, capivasertib, or a pharmaceutically acceptable salt thereof, is administered once daily under a continuous dosing schedule at a dosage from about 300 mg to about 650 mg. In some embodiments, capivasertib, or a pharmaceutically acceptable salt thereof, is administered twice daily (BID) under a continuous dosing schedule. In one aspect, capivasertib, or a pharmaceutically acceptable salt thereof, is administered under a continuous dosing schedule at a dosage from about 100 mg to about 800 mg twice daily. In another aspect, capivasertib, or a pharmaceutically acceptable salt thereof, is administered under a continuous dosing schedule at a dosage from about 150 mg to about 750 mg twice daily. In another aspect, capivasertib, or a pharmaceutically acceptable salt thereof, is administered under a continuous dosing schedule at a dosage from about 200 mg to about 700 mg twice daily. In another aspect, capivasertib, or a pharmaceutically acceptable salt thereof, is administered under a continuous dosing schedule at a dosage from about 225 mg to about 650 mg twice daily. In another aspect, capivasertib, or a pharmaceutically acceptable salt thereof, is administered under a continuous dosing schedule at a dosage from about 250 mg to about 650 mg twice daily. In another aspect, capivasertib, or a pharmaceutically acceptable salt thereof, is administered under a continuous dosing schedule at a dosage from about 300 mg to about 600 mg twice daily. In another aspect, capivasertib, or a pharmaceutically acceptable salt thereof, is administered under a continuous dosing schedule at a dosage from about 200 mg to about 300 mg twice daily. In another aspect, capivasertib, or a pharmaceutically acceptable salt thereof, is administered under a continuous dosing schedule at a dosage from about 300 mg to about 400 mg twice daily. In another aspect, capivasertib, or a pharmaceutically acceptable salt thereof, is administered under a continuous dosing schedule at a dosage from about 400 mg to about 500 mg twice daily. In another aspect, capivasertib, or a pharmaceutically acceptable salt thereof, is administered under a continuous dosing schedule at a dosage from about 500 mg to about 600 mg twice daily. In another aspect, capivasertib, or a pharmaceutically acceptable salt thereof, is administered under a continuous dosing schedule at a dosage from about 600 mg to about 700 mg twice daily. In another aspect, capivasertib, or a pharmaceutically acceptable salt thereof, is administered under a continuous dosing schedule at a dosage from about 700 mg to about 800 mg twice daily. In another aspect, capivasertib, or a pharmaceutically acceptable salt thereof, is administered under a continuous dosing schedule at a dosage of about 160 mg twice daily. In another aspect, capivasertib, or a pharmaceutically acceptable salt thereof, is administered under a continuous dosing schedule at a dosage of about 200 mg twice daily. In another aspect, capivasertib, or a pharmaceutically acceptable salt thereof, is administered under a continuous dosing schedule at a dosage of about 240 mg twice daily. In another aspect, capivasertib, or a pharmaceutically acceptable salt thereof, is administered under a continuous dosing schedule at a dosage of about 280 mg twice daily. In another aspect, capivasertib, or a pharmaceutically acceptable salt thereof, is administered under a continuous dosing schedule at a dosage of about 320 mg twice daily. In another aspect, capivasertib, or a pharmaceutically acceptable salt thereof, is administered under a continuous dosing schedule at a dosage of about 360 mg twice daily. In another aspect, capivasertib, or a pharmaceutically acceptable salt thereof, is administered under a continuous dosing schedule at a dosage of about 400 mg twice daily. In another aspect, capivasertib, or a pharmaceutically acceptable salt thereof, is administered under a continuous dosing schedule at a dosage of about 440 mg twice daily. In another aspect, capivasertib, or a pharmaceutically acceptable salt thereof, is administered under a continuous dosing schedule at a dosage of about 480 mg twice daily. In another aspect, capivasertib, or a pharmaceutically acceptable salt thereof, is administered under a continuous dosing schedule at a dosage of about 520 mg twice daily. In another aspect, capivasertib, or a pharmaceutically acceptable salt thereof, is administered under a continuous dosing schedule at a dosage of about 580 mg twice daily. In another aspect, capivasertib, or a pharmaceutically acceptable salt thereof, is administered under a continuous dosing schedule at a dosage of about 600 mg twice daily. In another aspect, capivasertib, or a pharmaceutically acceptable salt thereof, is administered under a continuous dosing schedule at a dosage of about 640 mg twice daily. In another aspect, capivasertib, or a pharmaceutically acceptable salt thereof, is administered under a continuous dosing schedule at a dosage of about 680 mg twice daily. In another aspect, capivasertib, or a pharmaceutically acceptable salt thereof, is administered under a continuous dosing schedule at a dosage of about 720 mg twice daily. In another aspect, capivasertib, or a pharmaceutically acceptable salt thereof, is administered under a continuous dosing schedule at a dosage of about 760 mg twice daily. In another aspect, capivasertib, or a pharmaceutically acceptable salt thereof, is administered under a continuous dosing schedule at a dosage of about 800 mg twice daily. In some embodiments, capivasertib, or a pharmaceutically acceptable salt thereof, is administered to the subject on an intermittent dosage schedule. Administering capivasertib, or a pharmaceutically acceptable salt thereof, on an intermittent dosage schedule can, for example, have greater effectiveness and/or tolerability than on a continuous dosing schedule. In one aspect, capivasertib, or a pharmaceutically acceptable salt thereof, is intermittently dosed on a 1 day on/6 days off schedule (i.e., capivasertib, or a pharmaceutically acceptable salt thereof, is administered for one day followed by a six-day holiday). In another aspect, capivasertib, or a pharmaceutically acceptable salt thereof, is intermittently dosed on a 2 days on/5 days off schedule (i.e., capivasertib, or a pharmaceutically acceptable salt thereof, is administered for two days followed by a five-day holiday). In another aspect, capivasertib, or a pharmaceutically acceptable salt thereof, is intermittently dosed on a 3 days on/4 days off schedule (i.e., capivasertib, or a pharmaceutically acceptable salt thereof, is administered for three days followed by a four-day holiday). In another aspect, capivasertib, or a pharmaceutically acceptable salt thereof, is intermittently dosed on a 4 days on/3 days off schedule (i.e., capivasertib, or a pharmaceutically acceptable salt thereof, is administered for four days followed by a three-day holiday). In another aspect, capivasertib, or a pharmaceutically acceptable salt thereof, is intermittently dosed on a 5 days on/2 days off schedule (i.e., capivasertib, or a pharmaceutically acceptable salt thereof, is administered for five days followed by a two-day holiday). In another aspect, capivasertib, or a pharmaceutically acceptable salt thereof, is intermittently dosed on a 6 days on/1 day off schedule (i.e., capivasertib, or a pharmaceutically acceptable salt thereof, is administered for six days followed by a one-day holiday). The dosing cycle of such embodiments would then repeat as long as tolerable and beneficial for the subject. In some embodiments, the dosing cycle is 7 days. In one aspect, the dosing cycle is 14 days. In another aspect, the dosing cycle is 21 days. In another aspect, the dosing cycle is 28 days. In another aspect, the dosing cycle is two months. In another aspect, the dosing cycle is six months. In another aspect, the dosing cycle is one year.

In some embodiments, the dosing cycle is 28 days, but capivasertib, or a pharmaceutically acceptable salt thereof, is not co-administered to the subject during the fourth week of the dosing cycle (i.e., there is a capivasertib, or a pharmaceutically acceptable salt thereof, drug holiday during the final week of the dosing cycle).

In some embodiments, capivasertib, or a pharmaceutically acceptable salt thereof, is administered once daily (QD) under an intermittent dosing schedule. In one aspect, capivasertib, or a pharmaceutically acceptable salt thereof, is administered once daily under an intermittent dosing schedule at a dosage from about 100 mg to about 900 mg. In another aspect, capivasertib, or a pharmaceutically acceptable salt thereof, is administered once daily under an intermittent dosing schedule at a dosage from about 150 mg to about 850 mg. In another aspect, capivasertib, or a pharmaceutically acceptable salt thereof, is administered once daily under an intermittent dosing schedule at a dosage from about 175 mg to about 800 mg. In another aspect, capivasertib, or a pharmaceutically acceptable salt thereof, is administered once daily under an intermittent dosing schedule at a dosage from about 200 mg to about 750 mg. In another aspect, capivasertib, or a pharmaceutically acceptable salt thereof, is administered once daily under an intermittent dosing schedule at a dosage from about 225 mg to about 725 mg. In another aspect, capivasertib, or a pharmaceutically acceptable salt thereof, is administered once daily under an intermittent dosing schedule at a dosage from about 250 mg to about 700 mg. In another aspect, capivasertib, or a pharmaceutically acceptable salt thereof, is administered once daily under an intermittent dosing schedule at a dosage from about 275 mg to about 675 mg. In another aspect, capivasertib, or a pharmaceutically acceptable salt thereof, is administered once daily under an intermittent dosing schedule at a dosage from about 300 mg to about 650 mg. In some embodiments, capivasertib, or a pharmaceutically acceptable salt thereof, is administered twice daily (BID) under an intermittent dosing schedule. In one aspect, capivasertib, or a pharmaceutically acceptable salt thereof, is administered under an intermittent dosing schedule at a dosage from about 100 mg to about 800 mg twice daily. In another aspect, capivasertib, or a pharmaceutically acceptable salt thereof, is administered under an intermittent dosing schedule at a dosage from about 150 mg to about 750 mg twice daily. In another aspect, capivasertib, or a pharmaceutically acceptable salt thereof, is administered under an intermittent dosing schedule at a dosage from about 200 mg to about 700 mg twice daily. In another aspect, capivasertib, or a pharmaceutically acceptable salt thereof, is administered under an intermittent dosing schedule at a dosage from about 225 mg to about 675 mg twice daily. In another aspect, capivasertib, or a pharmaceutically acceptable salt thereof, is administered under an intermittent dosing schedule at a dosage from about 250 mg to about 650 mg twice daily. In another aspect, capivasertib, or a pharmaceutically acceptable salt thereof, is administered under an intermittent dosing schedule at a dosage from about 300 mg to about 625 mg twice daily. In another aspect, capivasertib, or a pharmaceutically acceptable salt thereof, is administered under an intermittent dosing schedule at a dosage from about 200 mg to about 300 mg twice daily. In another aspect, capivasertib, or a pharmaceutically acceptable salt thereof, is administered under an intermittent dosing schedule at a dosage from about 300 mg to about 400 mg twice daily. In another aspect, capivasertib, or a pharmaceutically acceptable salt thereof, is administered under an intermittent dosing schedule at a dosage from about 400 mg to about 500 mg twice daily. In another aspect, capivasertib, or a pharmaceutically acceptable salt thereof, is administered under an intermittent dosing schedule at a dosage from about 500 mg to about 600 mg twice daily. In another aspect, capivasertib, or a pharmaceutically acceptable salt thereof, is administered under an intermittent dosing schedule at a dosage from about 600 mg to about 700 mg twice daily. In another aspect, capivasertib, or a pharmaceutically acceptable salt thereof, is administered under an intermittent dosing schedule at a dosage from about 700 mg to about 800 mg twice daily. In another aspect, capivasertib, or a pharmaceutically acceptable salt thereof, is administered under an intermittent dosing schedule at a dosage of about 160 mg twice daily. In another aspect, capivasertib, or a pharmaceutically acceptable salt thereof, is administered under an intermittent dosing schedule at a dosage of about 200 mg twice daily. In another aspect, capivasertib, or a pharmaceutically acceptable salt thereof, is administered under an intermittent dosing schedule at a dosage of about 240 mg twice daily. In another aspect, capivasertib, or a pharmaceutically acceptable salt thereof, is administered under an intermittent dosing schedule at a dosage of about 280 mg twice daily. In another aspect, capivasertib, or a pharmaceutically acceptable salt thereof, is administered under an intermittent dosing schedule at a dosage of about 320 mg twice daily. In another aspect, capivasertib, or a pharmaceutically acceptable salt thereof, is administered under an intermittent dosing schedule at a dosage of about 360 mg twice daily. In another aspect, capivasertib, or a pharmaceutically acceptable salt thereof, is administered under an intermittent dosing schedule at a dosage of about 400 mg twice daily. In another aspect, capivasertib, or a pharmaceutically acceptable salt thereof, is administered under an intermittent dosing schedule at a dosage of about 440 mg twice daily. In another aspect, capivasertib, or a pharmaceutically acceptable salt thereof, is administered under an intermittent dosing schedule at a dosage of about 480 mg twice daily. In another aspect, capivasertib, or a pharmaceutically acceptable salt thereof, is administered under an intermittent dosing schedule at a dosage of about 520 mg twice daily. In another aspect, capivasertib, or a pharmaceutically acceptable salt thereof, is administered under an intermittent dosing schedule at a dosage of about 580 mg twice daily. In another aspect, capivasertib, or a pharmaceutically acceptable salt thereof, is administered under an intermittent dosing schedule at a dosage of about 600 mg twice daily. In another aspect, capivasertib, or a pharmaceutically acceptable salt thereof, is administered under an intermittent dosing schedule at a dosage of about 640 mg twice daily. In another aspect, capivasertib, or a pharmaceutically acceptable salt thereof, is administered under an intermittent dosing schedule at a dosage of about 680 mg twice daily. In another aspect, capivasertib, or a pharmaceutically acceptable salt thereof, is administered under an intermittent dosing schedule at a dosage of about 720 mg twice daily. In another aspect, capivasertib, or a pharmaceutically acceptable salt thereof, is administered under an intermittent dosing schedule at a dosage of about 760 mg twice daily. In another aspect, capivasertib, or a pharmaceutically acceptable salt thereof, is administered under an intermittent dosing schedule at a dosage of about 800 mg twice daily.

### Perifosine

Perifosine has the following chemical structure:

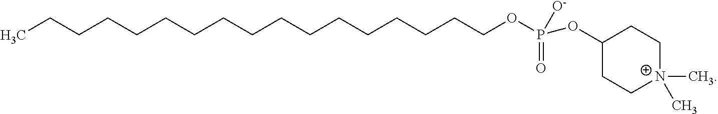 [SMILES: CCCCCCCCCCCCCCCCCOP(=O)([O-])OC1CC[N+](C)(C)CC1]

Perifosine is known by the chemical name 1,1-Dimethylpiperidinium-4-yl octadecyl phosphate. Perifosine is disclosed in U.S. Pat. No. 8,383,607.

### MK-2206

MK-2206 has the following chemical structure:

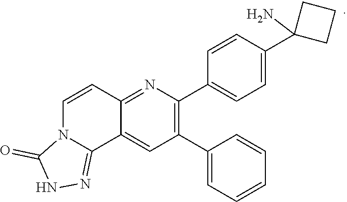 [SMILES: NC1(c2ccc(-c3nc4ccn5c(=O)[nH]nc5c4cc3-c3ccccc3)cc2)CCC1]

The free base of MK-2206 is known by the chemical name 8-[4-(1-Aminocyclobutyl)phenyl]-9-phenyl[1,2,4]triazolo[3,4-f][1,6]naphthyridin-3(2H)-one. MK-2206 is disclosed in WO2008070016.

### GSK690693

GSK690693 has the following chemical structure:

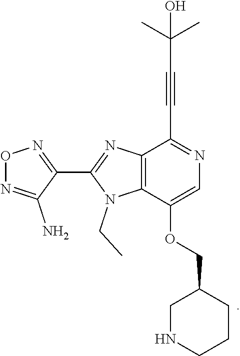 [SMILES: CCn1c(-c2nonc2N)nc2c(C#CC(C)(C)O)ncc(OC[C@H]3CCCNC3)c21]

The free base of GSK690693 is known by the chemical name 4-(2-(4-Amino-1,2,5-oxadiazol-3-yl)-1-ethyl-7-{[(3S)-3-piperidinylmethyl]oxy}-1H-imidazo[4,5-c]pyridin-4-yl)-2-methyl-3-butyn-2-ol. GSK690693 is disclosed in WO2007058850.

### Afuresertib

Afuresertib (GSK2110183) has the following chemical structure:

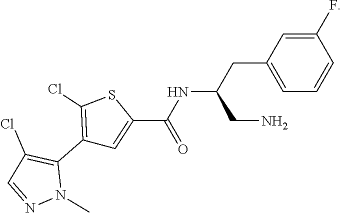 [SMILES: Cn1ncc(Cl)c1-c1cc(C(=O)N[C@H](CN)Cc2cccc(F)c2)sc1Cl]

The free base of afuresertib is known by the chemical name N-[(1S)-2-amino-1-[(3-fluorophenyl)methyl]ethyl]-5-chloro-4-(4-chloro-1-methyl-1H-pyrazol-5-yl)-2-thiophenecarboxamide.

Afuresertib is disclosed in WO2008098104.

### Uprosertib

Uprosertib (GSK2141795) has the following chemical structure:

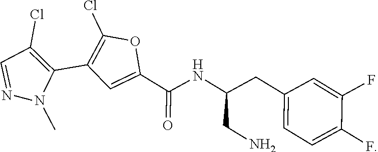 [SMILES: Cn1ncc(Cl)c1-c1cc(C(=O)N[C@H](CN)Cc2ccc(F)c(F)c2)oc1Cl]

The free base of uprosertib is known by the chemical name N-[(1S)-2-amino-1-[(3,4-difluorophenyl)methyl]ethyl]-5-chloro-4-(4-chloro-1-methyl-1H-pyrazol-5-yl)-2-furancarboxamide.

Uprosertib is disclosed in WO2008098104.

### Ipatasertib

Ipatasertib has the following chemical structure:

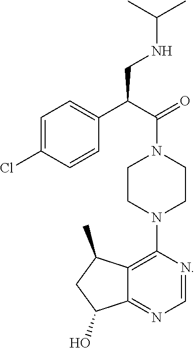 [SMILES: CC(C)NC[C@@H](C(=O)N1CCN(c2ncnc3c2[C@H](C)C[C@H]3O)CC1)c1ccc(Cl)cc1]

The free base of ipatasertib is known by the chemical name 2-(4-chlorophenyl)-1-(4-((5R,7R)-7-hydroxy-5-methyl-6,7-dihydro-5H-cyclopenta[d]pyrimidin-4-yl)piperazin-1-yl)-3-(isopropylamino)propan-1-one.

Ipatasertib is disclosed in WO2008006040.

### Further Embodiments

In an aspect there is provided an EGFR TKI for use in the treatment of cancer in a human patient, wherein the EGFR TKI is administered in combination with an AKT inhibitor. In embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising administering to the human patient a therapeutically effective amount of an EGFR TKI, wherein the EGFR TKI is administered in combination with a therapeutically effective amount of an AKT inhibitor. In embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising administering to the human patient a first amount of an EGFR TKI, and a second amount of an AKT inhibitor, where the first amount and the second amount together comprise a therapeutically effective amount. In embodiments, the cancer is lung cancer, such as NSCLC. In yet further embodiments, the NSCLC is an EGFR mutation-positive NSCLC.

In an aspect there is provided the use of an EGFR TKI in the manufacture of a medicament for the treatment of cancer in a human patient, wherein the EGFR TKI is administered in combination with an AKT inhibitor. In embodiments, the cancer is lung cancer, such as NSCLC. In yet further embodiments, the NSCLC is an EGFR mutation-positive NSCLC.

In an aspect there is provided a combination of an EGFR TKI and AKT inhibitor for use in the treatment of cancer in a human patient. In embodiments the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising administering to the human patient a combination of a therapeutically effective amount of an EGFR TKI and a therapeutically effective amount of an AKT inhibitor. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising administering to the human patient a first amount of an EGFR TKI, and a second amount of an AKT inhibitor, where the first amount and the second amount together comprise a therapeutically effective amount. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided the use of a combination of an EGFR TKI and AKT inhibitor in the manufacture of a medicament for treatment of cancer in a human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided a combination of osimertinib or a pharmaceutically acceptable salt thereof and AKT inhibitor for use in the treatment of cancer in a human patient, wherein the osimertinib, or pharmaceutically acceptable salt thereof, is administered to the human patient before the AKT inhibitor is administered to the human patient. In embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising administering to the human patient a combination of a therapeutically effective amount of osimertinib or a pharmaceutically acceptable salt thereof and a therapeutically effective amount of an AKT inhibitor, wherein the osimertinib, or pharmaceutically acceptable salt thereof, is administered to the human patient before the AKT inhibitor is administered to the human patient. In embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising administering to the human patient a first amount of an EGFR TKI, and a second amount of an AKT inhibitor, where the first amount and the second amount together comprise a therapeutically effective amount, wherein the osimertinib, or pharmaceutically acceptable salt thereof, is administered to the human patient before the AKT inhibitor is administered to the human patient. In embodiments, the cancer is lung cancer, such as NSCLC. In yet further embodiments, the NSCLC is an EGFR mutation-positive NSCLC.

In an aspect there is provided the use of a combination of osimertinib or a pharmaceutically acceptable salt thereof and an AKT inhibitor for the manufacture of a medicament for the treatment of cancer in a human patient, wherein the osimertinib, or pharmaceutically acceptable salt thereof, is administered to the human patient before the AKT inhibitor is administered to the human patient. In embodiments, the cancer is lung cancer, such as NSCLC. In yet further embodiments, the NSCLC is an EGFR mutation-positive NSCLC. In an aspect there is provided an EGFR TKI for use in the treatment of cancer in a human patient, wherein the treatment comprises the separate, sequential, or simultaneous administration of i) the EGFR TKI and ii) AKT inhibitor to the human patient. Where treatment is separate or sequential, the interval between the dose of EGFR TKI and the dose of AKT inhibitor may be chosen to ensure the production of a combined therapeutic effect.

A “therapeutic effect” encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

In embodiments, the administration of the EGFR TKI and the AKT inhibitor is sequential and the EGFR TKI is administered prior to the AKT inhibitor.

In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising the separate, sequential, or simultaneous administration of i) a therapeutically effective amount of an EGFR TKI and ii) a therapeutically effective amount of an AKT inhibitor to the human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided a method of treating cancer in a human patient in need of such a treatment comprising the separate, sequential, or simultaneous administration of i) a first amount of an EGFR TKI and ii) a second amount of an AKT inhibitor to the human patient, where the first amount and the second amount together comprise a therapeutically effective amount. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided use of an EGFR TKI in the manufacture of a medicament for the treatment of cancer in a human patient, wherein the treatment comprises the separate, sequential, or simultaneous administration of i) the EGFR TKI and ii) AKT inhibitor to the human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided an AKT inhibitor for use in the treatment of cancer in a human patient, wherein the AKT inhibitor is administered in combination with an EGFR TKI.

In an aspect there is provided an AKT inhibitor for use in the treatment of cancer in a human patient, wherein the treatment comprises the separate, sequential, or simultaneous administration of i) an AKT inhibitor and ii) an EGFR TKI to the human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided use of an AKT inhibitor in the manufacture of a medicament for the treatment of cancer in a human patient, wherein the treatment comprises the separate, sequential, or simultaneous administration of i) an EGFR TKI and ii) the AKT inhibitor to the human patient. In embodiments, the EGFR TKI is osimertinib or a pharmaceutically acceptable salt thereof. In further embodiments, the human patient is an EGFR TKI-naïve human patient. In further embodiments, the human patient has previously received EGFR TKI treatment. In further embodiments, the human patient has previously received osimertinib or a pharmaceutically acceptable salt thereof. In still further embodiments, the cancer is lung cancer, such as NSCLC. In yet further embodiments, the NSCLC is an EGFR mutation-positive NSCLC.

In an aspect there is provided a kit comprising:
a first pharmaceutical composition comprising an EGFR TKI and a pharmaceutically acceptable excipient; and
a second pharmaceutical composition comprising an AKT inhibitor and a pharmaceutically acceptable excipient.

In an aspect, there is provided an AKT inhibitor for use in the treatment of non-small cell lung cancer in a human patient, wherein the patient's disease has reached maximal response during or after previous EGFR TKI treatment. In embodiments, the human patient's disease has progressed during or after previous treatment with osimertinib or a pharmaceutically acceptable salt thereof.

In an aspect, there is provided osimertinib or a pharmaceutically acceptable salt thereof in the treatment of non-small cell lung cancer in a human patient, wherein the human patient's disease has progressed during or after previous treatment with a different EGFR TKI.

In an aspect, there is provided a method of treating non-small cell lung cancer in a human patient in need of such a treatment comprising administering to the human patient a therapeutically effective amount of an AKT inhibitor, wherein the patient's disease has progressed during or after previous EGFR TKI treatment. In embodiments, the human patient's disease has progressed during or after previous treatment with osimertinib or a pharmaceutically acceptable salt thereof.

In an aspect, there is provided the use of an AKT inhibitor in the manufacture of a medicament for the treatment of non-small cell lung cancer in a human patient, wherein the patient's disease has progressed during or after previous EGFR TKI treatment. In embodiments, the human patient's disease has progressed during or after previous treatment with osimertinib or a pharmaceutically acceptable salt thereof.

### Examples

The specific Examples below, with reference to the accompanying Figures, are provided for illustrative purposes only and are not to be construed as limiting the teachings herein.

PC9 is a cell line derived from human lung adenocarcinoma harbouring the activating mutation in EGFR del E746_A750 (Ex19-del). HCC-827 is a cell line derived from human lung adenocarcinoma harbouring the activating mutation in EGFR E746-A750 (Ex19del). Both cell lines were obtained from ATCC. LC-F-12 is a cell line derived from human lung adenocarcinoma harbouring the activating point mutations in EGFR L858R and in PIKC3A E545K was obtain from Xentech. MR131 is an in-house PDX derived from human lung adenocarcinoma harbouring the activating point mutations in EGFR L858R and PTEN D326H. CTG-2939 is a PDX derived from human lung adenocarcinoma harbouring the activating mutation in EGFR E746-A750 (Ex19del) and a PTEN Deep deletion, CTG-2180 is a PDX derived from human lung adenocarcinoma harbouring the activating mutation in EGFR L747_T751del (Ex19del) and a PTEN frameshift at C304, both models are available at Champions Oncology.

Unless otherwise stated, all reagents are commercially available and were used as supplied.

Example 1: PIK3CA Activating Mutations Drive Resistance to Osimertinib In Vitro, which can be Overcome by Combination Treatment with Capivasertib.

To preclinically validate the hypothesis that PIK3CA-activating mutations drive resistance to osimertinib, we introduced the PIK3CA^{H1047R} and PIK3CA^{E453K} variants in lung cancer cell line models using a CRISPR/Cas9 technology. As the efficiency of knock-ins using this technology is usually low, only a small proportion of cells was expected to be genetically modified, thus mimicking the emergence of a co-occurring resistance mutation in a tumour. This heterogeneous cell pool was then cultured under the selection pressure of osimertinib (100 nM) for 3 weeks to generate an osimertinib-resistant cell pool for downstream analyses. DNA sequencing (NGS and SANGER) of CRISPR cell pools at the end of osimertinib selection confirmed selective outgrowth of the PIK3CA H1047R and PIK3CA E453K-positive cells, indicating that the inserted PIK3CA mutations conferred resistance to osimertinib.

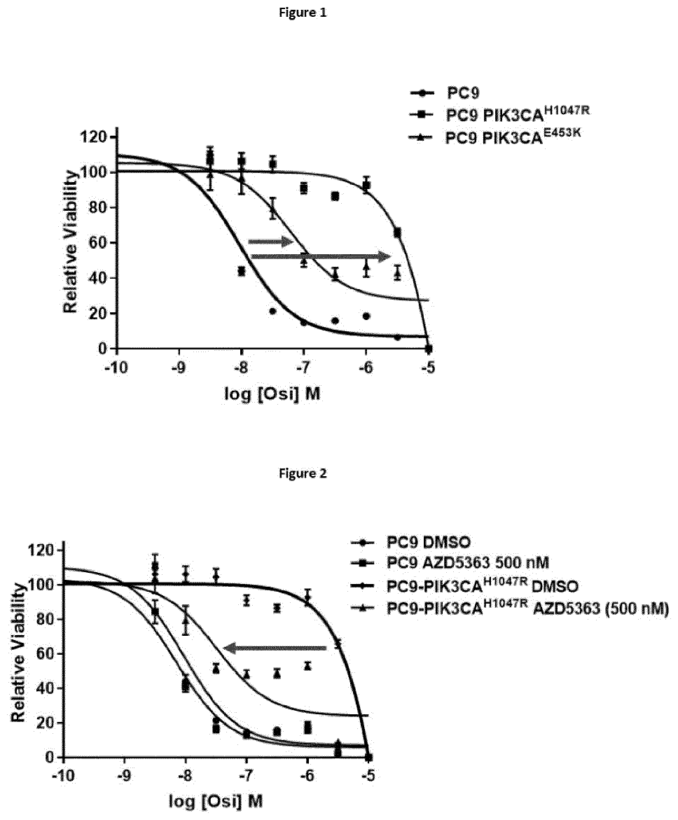

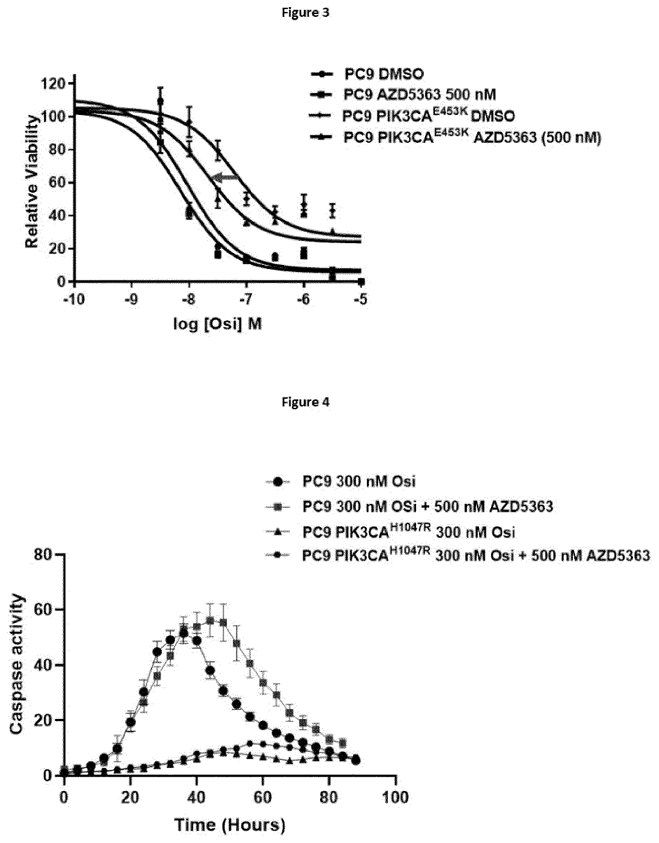

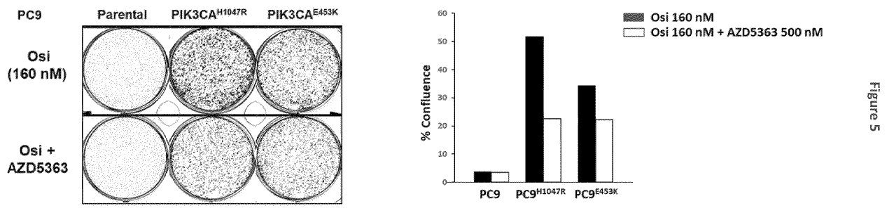

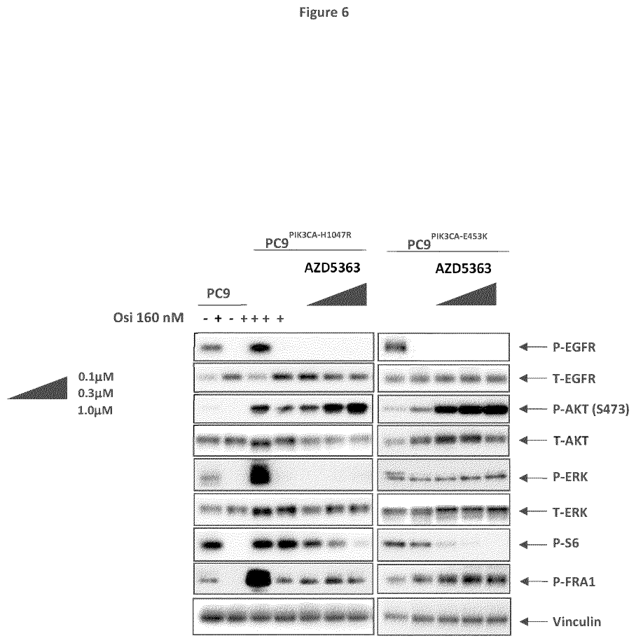

PIK3CAm-induced resistance to osimertinib and rescue by combination with capivasertib was analysed in more detail by different experimental approaches including:
a) Cell viability: the viability of the osimertinib-resistant PC9-PIK3CAm cell pool was compared to the parental PC9 cells upon treatment with osimertinib alone or in combination with capivasertib. PIK3CAm-induced resistance to osimertinib was associated to increased EC₅₀ for osimertinib and could be partially rescued by co-treatment with capivasertib (FIGS. 1-3: Cell-TiterGlo® assay in PC9-PIK3CA^{H1047R} and PC9-PIK3CA^{E453K} treated with osimertinib 3 nM-10 μM alone or in combination with capivasertib 500 nM for 6 days).
b) Caspase 3/7 activation assay: osimertinib-induced apoptotic response in PC9-PIK3CAm cells was compared to parental PC9 cell by Caspase-Glo® 3/7 Assay, a luminescent assay that measures intracellular caspase-3 and -7 activities. PIK3CAm-induced resistance to osimertinib was associated to diminished apoptotic response and could not be restored by co-treatment with capivasertib (FIG. 4).
c) Clonogenic assay: Proliferative properties of PC9-PIK3CAm cells was compared to parental PC9. Cells were plated at low density (1.5×10³) in 6 well plates and treated with osimertinib (160 nM) alone or in combination with capivasertib (500 nM). Cells were stained with Crystal violet at the end of the treatment (8 days) and cell density (% surface) quantified by ImageJ. As shown in FIG. 5, PC9-PIK3Cam cells developed resistance to osimertinib that can be partially rescued by combination with capivasertib.
d) Intracellular changes by WB analysis. Protein analysis by western blot showed an increase in the basal level of pAKT, pERK and pS6 in the PC9-PIK3CAm CRISPR cell pool when compared to parental PC9 cells, indicating activation of downstream PI3K/AKT and MAPK signalling pathways in the osimertinib-resistant cells. Treatment with osimertinib resulted in downregulation of P-EGFR levels and MAPK signalling in both parental and PC9-PIK3CAm cells. However, in PC9-PIK3CAm cells levels of P-AKT and P-S6 was refractory to osimertinib treatment and could be partially downregulated by co-treatment with osimertinib+capivasertib in a dose-dependent manner (FIG. 6, PC9 and PC9 PIK3CAm cells treated with 160 nM osimertinib alone or in combination with 100 nM-300 nM-1 μM capivasertib for 4 h).
   Example 2: PTEN-Loss Drive Resistance to Osimertinib In Vitro, which can be Overcome by Combination Treatment with Capivasertib

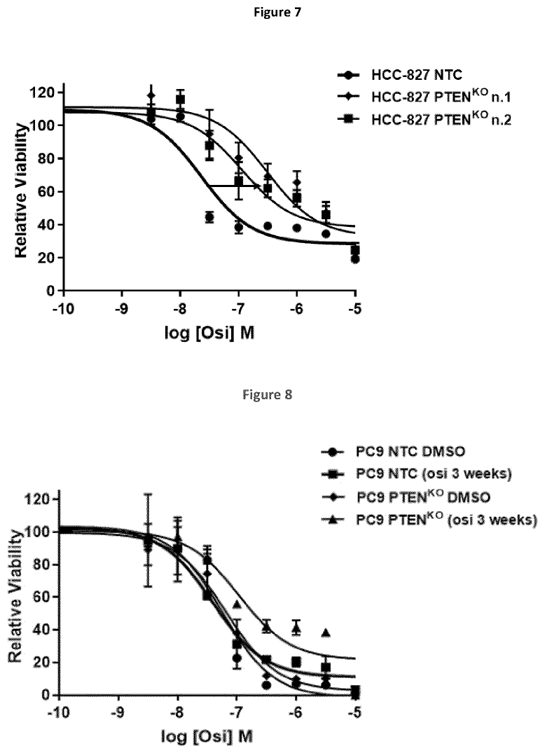

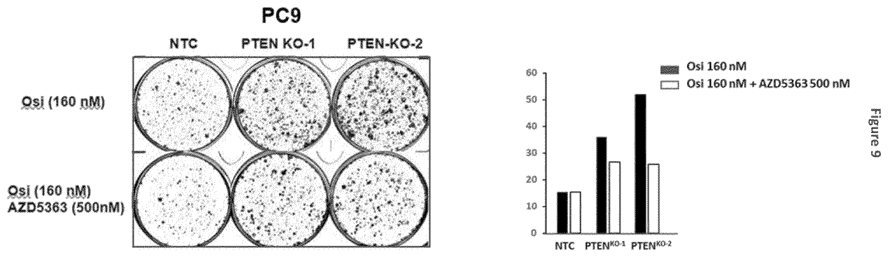

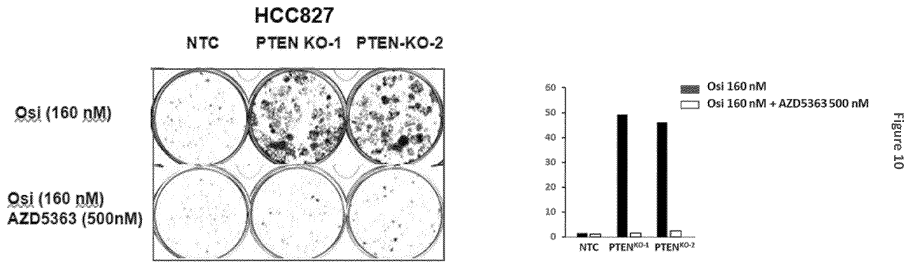

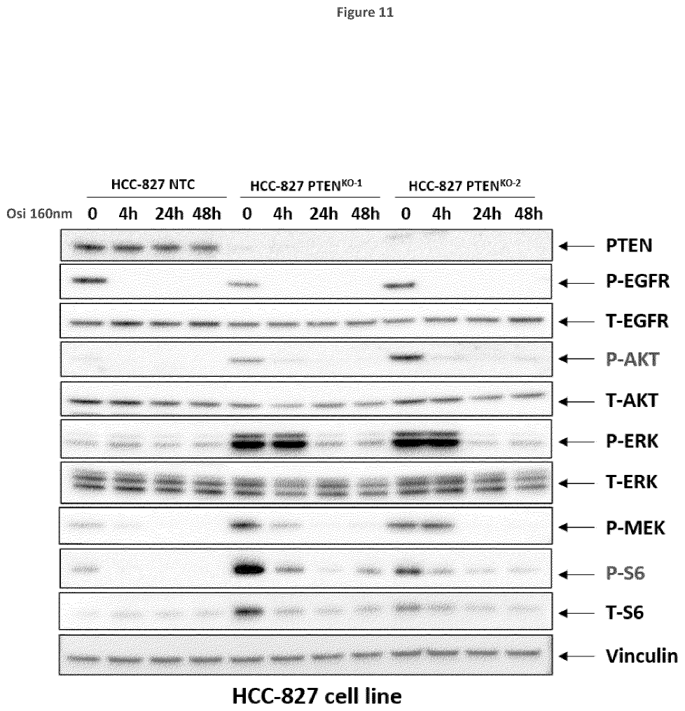

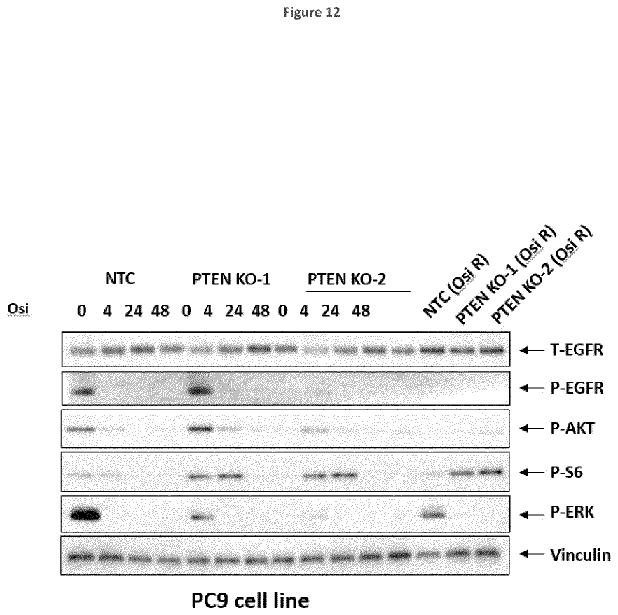

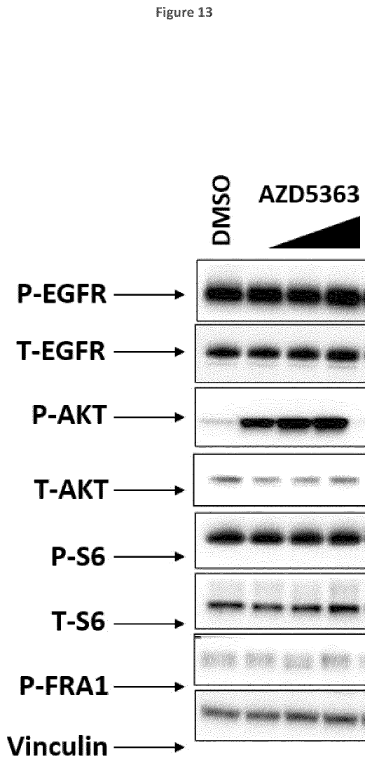

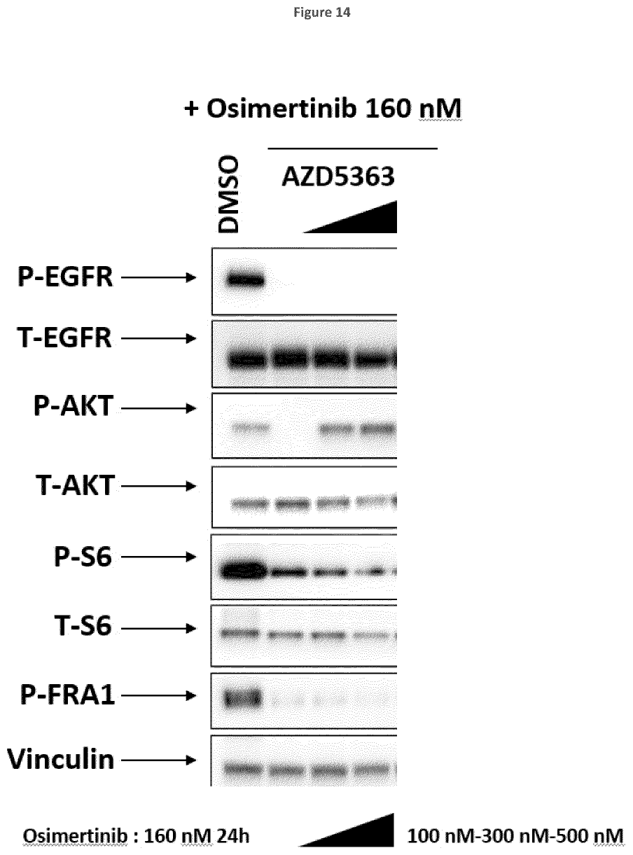

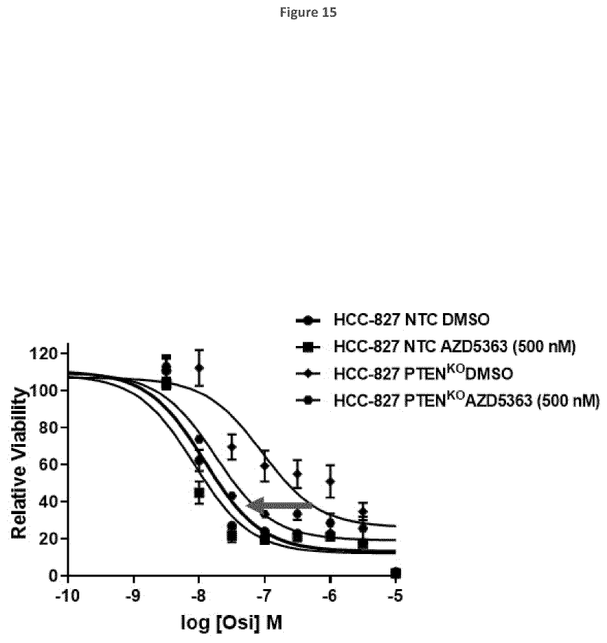

To preclinically validate the hypothesis that PTEN loss drives resistance to osimertinib, PTEN was depleted by CRSIPR KO in 2 NSCLC cell lines (PC9 and HCC-827). DNA sequencing and WB analysis confirmed depletion of PTEN in the generated PC9 and HCC-827 PTEN^{KO} (“PTEN knock out”) cell lines and resistance to osimertinib and rescue by combination with capivasertib was evaluated by:
a) Cell viability: Sensitivity to osimertinib of HCC-827 PTEN KO and PC9 PTEN^{KO} cell lines was compared to parental cells by CTG (osimertinib 3 nM-10 μM treatment for 6 days). Two independent HCC-827 PTEN^{KO} cell lines displayed resistance to osimertinib when compared to HCC-827 parental cells as indicated by increased EC₅₀ for osimertinib (FIG. 7). PC9 cells PTEN^{KO} displayed similar sensitivity to osimertinib when compared to parental PC9 cells and resistance manifested later upon osimertinib treatment for 2-3 weeks (FIG. 8); in this PC9 model resistance is associated with increased EC₅₀ in PC9 PTEN^{KO} cells treated for 3 weeks with 160 nM osimertinib when compared to PC9 cells that underwent same osimertinib exposure (PC9 NTC osimertinib 3 weeks).
b) Clonogenic assay: proliferative properties of HCC-827 PTEN^{KO} and PC9 PTEN^{KO} cell lines treated with 160 nM osimertinib were compared to parental cells in a long term assay. 1.5×10³ cells were plated at low density in 6 well plates, treated with 160 nM osimertinib and cell density (% surface) calculated at the end of the treatment. After 3 weeks, HCC-827 PTEN^{KO} cells displayed higher cell density (20 X) when compared to parental HCC-827, indicating that depletion of PTEN resulted in osimertinib resistance. Co-treatment with 500 nM of capivasertib resulted in partial rescue of osimertinib resistance (FIG. 9). In the PC9 model, a milder resistance phenotype was observed with cell density only 2× higher in PC9 PTEN^{KO} when compared to parental PC9 cells (FIG. 10).
c) Intracellular changes by WB analysis. Protein analysis by western blot showed elevated levels of P-AKT and P-S6 in both PC9 PTEN^{KO} and HCC-827 PTEN^{KO} when compared to parental cells. In HCC-827 PTEN^{KO} also elevated RAS-MAPK signalling was observed, although not predicted (FIG. 11). In PC9 PTEN^{KO} cells treated for 3 weeks with osimertinib 160 nM, persistent p-S6 levels were observed (FIG. 12). A dose-dependent downregulation of P-S6 levels was observed with capivasertib in combination with osimertinib (FIGS. 13 and 14).
d) Additional observations:
   In the HCC-827 in vitro model, resistance to osimertinib could be rescued by combination with capivasertib (FIG. 15, CTG assay), consistent with the hypothesis that PTEN loss drives resistance.
   In the PC9 in vitro model, the combination osimertinib+capivasertib could mildly sensitize cells to osimertinib. (FIG. 16, clonogenic assay).

### Example 3: In-Vivo Xenograft Models

Further in-vivo models using patient derived cell lines were used to investigate combination activity.

a) PC9 PIKC3A^{H1047} and PC9 PIKC3A^{E453K} cell lines were cultured in RPMI1640 supplemented with 10% FCS and cultured in a humidified incubator with 5% CO₂ at 37° C. PC9 PIKC3A^{H1047} and PC9 PIKC3A^{E453K} xenografts were established by subcutaneous implantation of 5×10⁶, cells per animal, in 100 μl of cell suspension including 50% matrigel, into the flank of female NOD/SCID mice. All mice were older than 6 weeks at the time of cell implant. Tumour growth was monitored twice weekly by bilateral calliper measurements and tumour volume calculated using the formula TV (mm3)=[length (mm)×width (mm)2]×0.5, where the length and the width are the longest and the shortest diameters of the tumour.

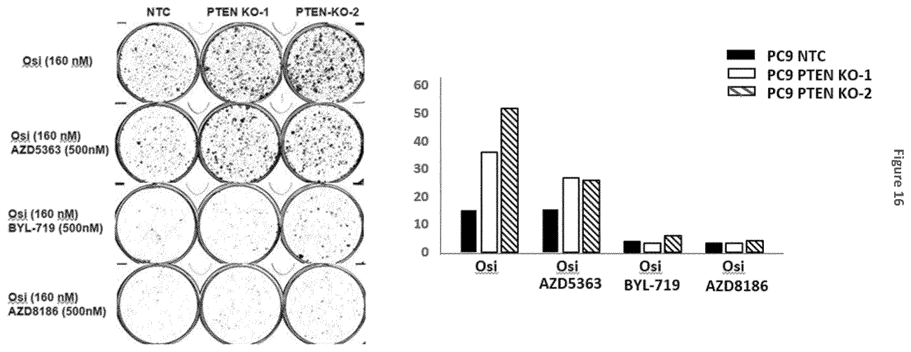

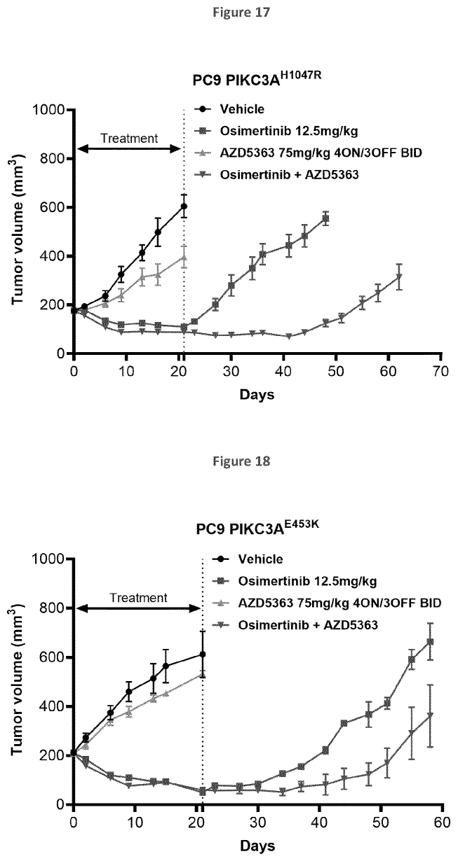

PC9 PIKC3A^{H1047} AND PC9 PIKC3A^{E453} are CRISPR engineered cell lines. FIGS. 17 and 18 show that the combination of an EGFR TKI and an AKT inhibitor enhance response on treatment and delay outgrowth when treatment is stopped.

b) LC-F-12 tumour fragments from donor mice inoculated with primary human lung cancer tissues were harvested and inoculated subcutaneously into the flank of athymic nude female mice. Tumour growth was monitored twice weekly by bilateral calliper measurements and tumour volume calculated using the formula TV (cm3)=[length (cm)×width (cm)2]×0.5, where the length and the width are the longest and the shortest diameters of the tumour.

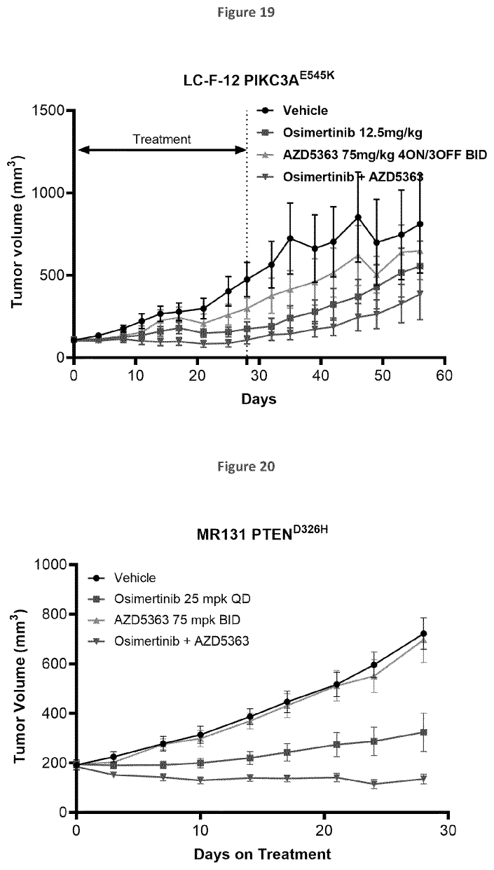

LC-F-12 is a PDX model derived from a TKI naïve patient. FIG. 19 shows that the combination of an EGFR TKI and AKT inhibitor enhance response on treatment and delay outgrowth when treatment is stopped.

c) MR131 and CTG-2939 tumour fragments from donor mice inoculated with primary human lung cancer tissues were harvested and inoculated subcutaneously into the flank of NSG female mice. Tumour growth was monitored twice weekly by bilateral calliper measurements and tumour volume calculated using the formula TV (cm3)=[length (cm)×width (cm)2]×0.5, where the length and the width are the longest and the shortest diameters of the tumour.

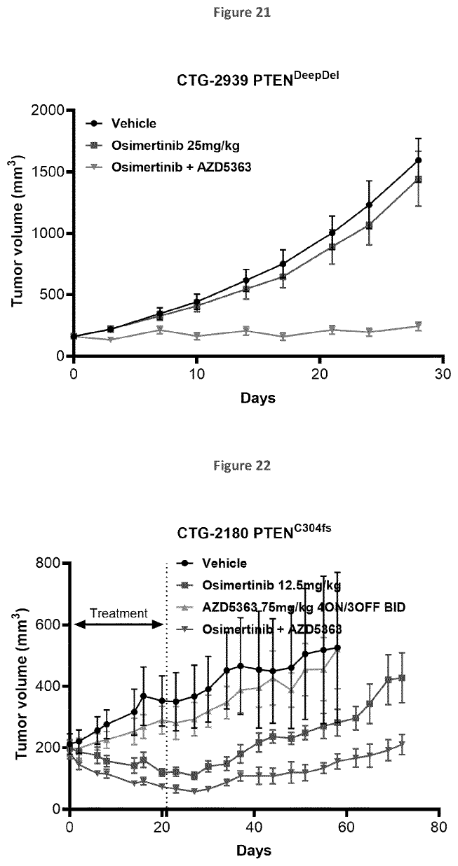

FIG. 20 shows that in MR131, a model of acquired resistance through PTEN loss, the combination of EGFR TKI and AKT inhibitor induces tumour stasis. Similarly, FIG. 21 shows that in CTG-2939, a model of acquired resistance through PTEN loss, the combination demonstrates superior activity than EGFR TKI monotherapy and induces tumour stasis.

d) CTG-2180 tumour fragments from donor mice inoculated with primary human lung cancer tissues were harvested and inoculated subcutaneously into the flank of athymic nude female mice. Tumour growth was monitored twice weekly by bilateral calliper measurements and tumour volume calculated using the formula TV (cm3)=[length (cm)×width (cm)2]×0.5, where the length and the width are the longest and the shortest diameters of the tumour.

FIG. 22 shows that in CTG-2180, a PDX model derived from a TKI naïve patient, the combination of EGFR TKI and AKT inhibitor induces superior tumour regression than EGFR TKI monotherapy.

e) PC9 PTEN-KO and HCC827 PTEN-KO cell lines were cultured in RPMI1640 supplemented with 10% FCS and cultured in a humidified incubator with 5% CO₂ at 37° C. PC9 PTEN-KO and HCC827 PTEN-KO xenografts were established by subcutaneous implantation of 5×10⁶, cells per animal, in 100 μL of cell suspension including 50% matrigel, into the flank of female NOD/SCID and nude mice respectively. All mice were older than 6 weeks at the time of cell implant. Tumour growth was monitored twice weekly by bilateral calliper measurements and tumour volume calculated using the formula TV (mm3)=[length (mm)×width (mm)2]×0.5, where the length and the width are the longest and the shortest diameters of the tumour.

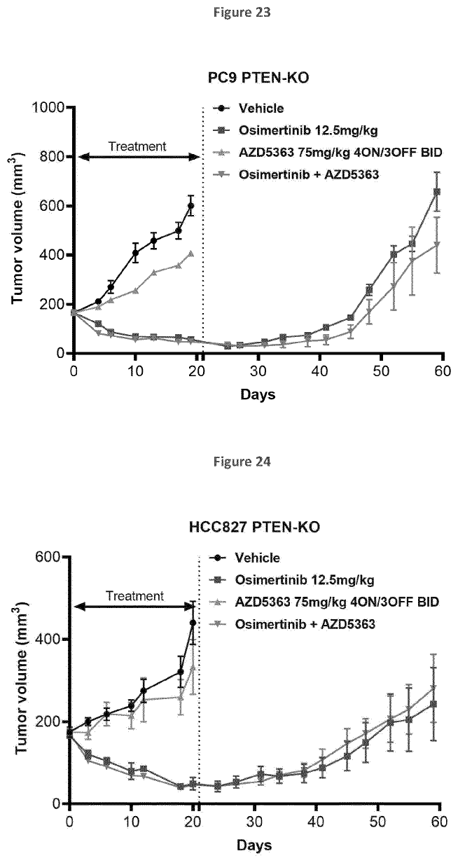

FIG. 23 shows in vivo, in the PTEN KO CRISPR engineered models there is no evidence of a combination benefit in between osimertinib and capivasertib. FIG. 24 shows In vivo, the degree of resistance to Osimertinib in the PC9/HCC827 PTEN KO models is at best very modest, so there is therefore limited opportunity to detect a combination benefit.

1. An EGFR TKI for use in the treatment of cancer in a human patient, wherein the EGFR TKI is administered in combination with an AKT inhibitor.

2. An EGFR TKI for use as claimed in claim 1, wherein the administration of the EGFR TKI and the AKT inhibitor is separate, sequential, or simultaneous.

3. An EGFR TKI for use as claimed in claim 1 or claim 2, wherein the EGFR TKI is a compound of Formula (I): 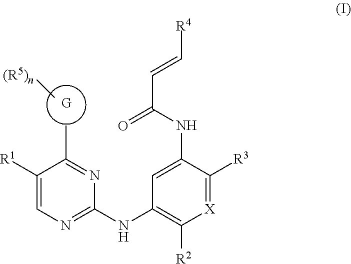
wherein:
G is selected from 4,5,6,7-tetrahydropyrazolo[1,5-a]pyridin-3-yl, indol-3-yl, indazol-1-yl, 3,4-dihydro-1H-[1,4]oxazino[4,3-a]indol-10-yl, 6,7,8,9-tetrahydropyrido[1,2-a]indol-10-yl, 5,6-dihydro-4H-pyrrolo[3,2,1-ij]quinolin-1-yl, pyrrolo[3,2-b]pyridin-3-yl and pyrazolo[1,5-a]pyridin-3-yl;
R¹ is selected from hydrogen, fluoro, chloro, methyl and cyano;
R² is selected from methoxy, trifluoromethoxy, ethoxy, 2,2,2-trifluoroethoxy and methyl;
R³ is selected from (3R)-3-(dimethylamino)pyrrolidin-1-yl, (3S)-3-(dimethyl-amino)pyrrolidin-1-yl, 3-(dimethylamino)azetidin-1-yl, [2-(dimethylamino)ethyl]-(methyl)amino, [2-(methylamino)ethyl](methyl)amino, 2-(dimethylamino)ethoxy, 2-(methylamino)ethoxy, 5-methyl-2,5-diazaspiro[3.4]oct-2-yl, (3aR,6aR)-5-methylhexa-hydro-pyrrolo[3,4-b]pyrrol-1 (2H)-yl, 1-methyl-1,2,3,6-tetrahydropyridin-4-yl, 4-methylpiperizin-1-yl, 4-[2-(dimethylamino)-2-oxoethyl]piperazin-1-yl, methyl[2-(4-methylpiperazin-1-yl)ethyl]amino, methyl[2-(morpholin-4-yl)ethyl]amino, 1-amino-1,2,3,6-tetrahydropyridin-4-yl and 4-[(2S)-2-aminopropanoyl]piperazin-1-yl;
R⁴ is selected from hydrogen, 1-piperidinomethyl and N,N-dimethylaminomethyl;
R⁵ is independently selected from methyl, ethyl, propyl, 2,2-difluoroethyl, 2,2,2-trifluoroethyl, fluoro, chloro and cyclopropyl;
X is CH or N; and
n is 0, 1 or 2;
or a pharmaceutically acceptable salt thereof.

4. An EGFR TKI for use as claimed in claim 3, wherein G is selected from indol-3-yl and indazol-1-yl; R¹ is selected from hydrogen, fluoro, chloro, methyl and cyano; R² is selected from methoxy and 2,2,2-trifluoroethoxy; R³ is selected from [2-(dimethylamino)ethyl]-(methyl)amino, [2-(methylamino)ethyl](methyl)amino, 2-(dimethylamino)ethoxy and 2-(methylamino)ethoxy; R⁴ is hydrogen; R⁵ is selected from methyl, 2,2,2-trifluoroethyl and cyclopropyl; X is CH or N; and n is 0 or 1; or a pharmaceutically acceptable salt thereof.

5. An EGFR TKI for use as claimed in claim 1 or claim 2, wherein the EGFR TKI is selected from the group consisting of osimertinib or a pharmaceutically acceptable salt thereof, AZD3759 or a pharmaceutically acceptable salt thereof, lazertinib or a pharmaceutically acceptable salt thereof, abivertinib or a pharmaceutically acceptable salt thereof, alflutinib or a pharmaceutically acceptable salt thereof, afatinib or a pharmaceutically acceptable salt thereof, CX-101 or a pharmaceutically acceptable salt thereof, HS-10296 or a pharmaceutically acceptable salt thereof, BPI-7711 or a pharmaceutically acceptable salt thereof, dacomitinib or a pharmaceutically acceptable salt thereof, icotinib or a pharmaceutically acceptable salt thereof, gefitinib or a pharmaceutically acceptable salt thereof and erlotinib or a pharmaceutically acceptable salt thereof.

6. An EGFR TKI for use as claimed in claim 5, wherein the EGFR TKI is selected from the group consisting of osimertinib or a pharmaceutically acceptable salt thereof, AZD3759 or a pharmaceutically acceptable salt thereof, alflutinib or a pharmaceutically acceptable salt thereof, HS-10296 or a pharmaceutically acceptable salt thereof, and lazertinib or a pharmaceutically acceptable salt thereof.

7. An EGFR TKI for use as claimed in claim 6, wherein the EGFR TKI is osimertinib or a pharmaceutically acceptable salt thereof.

8. An EGFR TKI for use as claimed in any of the previous claims, wherein the AKT inhibitor is selected from the group consisting of miransertib (ARQ-092) or a pharmaceutically acceptable salt thereof, BAY1125976 or a pharmaceutically acceptable salt thereof, borussertib or a pharmaceutically acceptable salt thereof, AT7867 or a pharmaceutically acceptable salt thereof, CCT128930 or a pharmaceutically acceptable salt thereof, A-674563 or a pharmaceutically acceptable salt thereof, PHT-427 or a pharmaceutically acceptable salt thereof, Akti-1/2 or a pharmaceutically acceptable salt thereof, AT13148 or a pharmaceutically acceptable salt thereof, SC79 or a pharmaceutically acceptable salt thereof, capivasertib or a pharmaceutically acceptable salt thereof, miltefosine or a pharmaceutically acceptable salt thereof, perifosine or a pharmaceutically acceptable salt thereof, MK-2206 or a pharmaceutically acceptable salt thereof, RX-0201 or a pharmaceutically acceptable salt thereof, erucylphosphocholine or a pharmaceutically acceptable salt thereof, PBI-05204 or a pharmaceutically acceptable salt thereof, GSK690693 or a pharmaceutically acceptable salt thereof, afuresertib (GSK2110183) or a pharmaceutically acceptable salt thereof, uprosertib (GSK2141795) or a pharmaceutically acceptable salt thereof, XL-418 or a pharmaceutically acceptable salt thereof and ipatasertib (GDC-0068) or a pharmaceutically acceptable salt thereof.

9. An EGFR TKI for use as claimed in any of the previous claims, wherein the cancer is non-small cell lung cancer.

10. An EGFR TKI for use as claimed in claim 9, wherein the non-small cell lung cancer is an EGFR mutation-positive non-small cell lung cancer.

11. An EGFR TKI for use as claimed in claim 10, wherein the EGFR mutation-positive non-small cell lung cancer comprises an activating mutation in EGFR selected from exon 19 deletions and L858R substitution mutations.

12. An EGFR TKI for use as claimed in claim 10 or claim 11, wherein the EGFR mutation-positive non-small cell lung cancer comprises a T790M mutation.

13. An EGFR TKI for use as claimed in any one of claims 1 to 11, wherein the human patient is an EGFR TKI-naïve human patient.

14. An EGFR TKI for use as claimed in any of the previous claims, wherein the cancer comprises a PIK3CA mutation.

15. An EGFR TKI for use as claimed in any of the previous claims, wherein the cancer is PTEN deficient.

16. An EGFR TKI for use as claimed in any one of claim 1 to 12, 14 or 15, wherein the human patient's disease has progressed during or after previous EGFR TKI treatment.

17. An EGFR TKI for use as claimed in claim 16, wherein the EGFR TKI is osimertinib or a pharmaceutically acceptable salt thereof and the human patient's disease has progressed during or after previous treatment with a different EGFR TKI.

18. The use of an EGFR TKI in the manufacture of a medicament for the treatment of cancer in a human patient, wherein the EGFR TKI is administered in combination with an AKT inhibitor.

19. A method of treating cancer in a human patient in need of such a treatment, comprising administering to the human patient a therapeutically effective amount of an EGFR TKI, wherein the EGFR TKI is administered in combination with a therapeutically effective amount of an AKT inhibitor.

20. A method of treating cancer in a human patient in need of such a treatment, comprising administering to the human patient a first amount of an EGFR TKI, and a second amount of an AKT inhibitor, where the first amount and the second amount together comprise a therapeutically effective amount.

21. A pharmaceutical composition comprising an EGFR TKI, an AKT inhibitor and a pharmaceutically acceptable excipient.

22. An AKT inhibitor for use in the treatment of cancer in a human patient, wherein the AKT inhibitor is administered in combination with an EGFR TKI.

23. An AKT inhibitor for use in the treatment of cancer as claimed in claim 22, where the EGFR TKI is osimertinib or a pharmaceutically acceptable salt thereof.

24. An AKT inhibitor for use in the treatment of non-small cell lung cancer as claimed in claim 22 or claim 23, where the AKT inhibitor is capivasertib or a pharmaceutically acceptable salt thereof.
